(19) United States
(12) Patent Application Publication
(10) Pub. No.: US 2026/0069590 A1
(43) Pub. Date: Mar. 12, 2026

(54) METHOD FOR TREATING NONTUBERCULOUS MYCOBACTERIAL INFECTION

(71) Applicant: MannKind Corporation, Danbury, CT (US)

(72) Inventors: Thomas Hofmann, Doylestown, PA (US); Michael Castagna, Danbury, CT (US)

(21) Appl. No.: 19/109,310

(22) PCT Filed: Sep. 5, 2023
(86) PCT No.: PCT/US2023/032002

§ 371 Date: Mar. 6, 2025

Related U.S. Application Data
(60) Provisional application No. 63/374,720, filed on Sep. 6, 2022.
(60) Provisional application No. 63/438,987, filed on Jan. 13, 2023.
(60) Provisional application No. 63/449,908, filed on Mar. 3, 2023.

(51) Int. Cl. A61K 31/498; A61K 9/00; A61K 31/7036; A61K 47/10; A61P 31/04

(52) U.S. Cl. CPC A61K 31/498; A61K 9/0078; A61K 31/7036; A61K 47/10; A61P 31/04

(57) ABSTRACT

The present invention relates to an inhalable pharmaceutical composition comprising clofazimine, or a pharmaceutically acceptable derivative of clofazimine, a clofazimine salt or a polymorph of clofazimine, or combination thereof, and a pharmaceutically acceptable carrier and/or excipient for use in the treatment or prophylaxis of a nontuberculous bacterial infection of the lungs, wherein clofazimine is in an amount of 1 mg to 20 mg wt % in the composition, and wherein the inhalable pharmaceutical composition is provided by inhalation in an effective daily dose of up to 90 mg of clofazimine. There is also provided pharmaceutical combinations comprising clofazimine in the form of an aerosol for pulmonary inhalation for administration alone, or concomitantly with other drugs as combination therapy in the treatment and/or prophylaxis of pulmonary infections caused by mycobacteria and other gram-positive bacteria, and of pulmonary fungal infections.

### RELATED APPLICATIONS

The present application claims benefit of priority to U.S. Provisional Patent Application No. 63/374,720, filed on Sep. 6, 2022, U.S. Provisional Patent Application No. 63/438,987, filed on Jan. 13, 2023, and U.S. Provisional Patent Application No. 63/449,908, filed on Mar. 3, 2023.

### TECHNICAL FIELD

Disclosed herein are methods and compositions for the treatment and prophylaxis of nontuberculous bacterial infections. In particular, the method comprises the administration of inhalable compositions for aerosolization, including, clofazimine in a solution, suspension, or dry powders for inhalation, administered by nebulization or oral inhalation to subjects in need of treatment.

### BACKGROUND

Nontuberculous mycobacterial (NTM) lung disease is a serious infection that is caused by bacteria common in the environment that can lead to a reduction in lung function, cough, fatigue, and quality of life. It is estimated that approximately 86,000 people in the U.S. are living with NTM lung disease, and it is on the rise growing 8% each year with women, the elderly, and those with underlying lung conditions at greatest risk.

Clofazimine is one of the three principal drugs recommended by the World Health Organization for the treatment of leprosy which is caused by Mycobacterium leprae and has been increasingly used for the treatment of other mycobacterial infections such as drug resistant tuberculosis and infections caused by nontuberculous mycobacteria (NTM) in recent years. Clofazimine has been delivered primarily in the form of oral capsules.

Clofazimine is an extremely hydrophobic riminophenazine antibiotic (Log P=7.66) with anti-mycobacterial and anti-inflammatory activities and was originally described in 1957. Its structural formula is as follows:

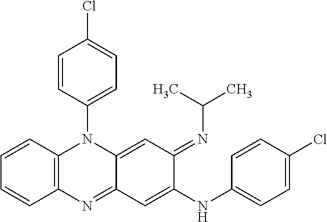 [SMILES: CC(C)/N=c1\cc2n(-c3ccc(Cl)cc3)c3ccccc3nc-2cc1Nc1ccc(Cl)cc1]

The exact mechanism through which clofazimine exerts its antimicrobial effect is unknown. However, it is known to bind preferentially to mycobacterial DNA, thereby inhibiting DNA replication and cell growth. Other suggested mechanisms of action include membrane damage/destabilization, generation of membrane-destabilizing lysophospholipids, interference of potassium transport, and/or intracellular redox cycling. While impressively active against Mycobacterium tuberculosis (MTB) in vitro, including multidrug-resistant strains, clofazimine, until recently, was generally considered to be ineffective in the treatment of pulmonary tuberculosis (see, for example, Cholo M et al., J Antimicrob Chemother, 2012 February, 67 (2): 290-8).

Clofazimine has been classified as a Biopharmaceutics Classification System (BCS) class II drug as it is practically insoluble in water and shows high membrane permeability. To overcome the problems associated with poor oral absorption and poor bioavailability of drugs, various strategies have been applied such as micronization, nanonization, supercritical fluid re-crystallization, spray freeze drying into liquid, solid dispersions and solutions in optimizing oral dosage forms.

Being classified as a BCS class II drug, clofazimine is generally considered an ideal candidate for the formulation into solid dispersions for improvement of oral bioavailability (see, for example, Bhusnure et al. IJRPC 2014, 4(4), 906-918). In line with this, because of its lipophilicity, clofazimine is generally administered as a microcrystalline suspension in an oil-wax base to improve oral absorption. The absorption in humans after oral administration varies considerably (45-62%). Adverse effects of clofazimine are dose related and primarily affect the skin, eyes, gastrointestinal tract, and QT elongation. Side effects include the development of reddish-brown discoloration of the skin and conjunctiva and are gradually reversible on cessation. They are the result of chronic systemic accumulation.

Mycobacterium is a genus Actinobacteria, with its own genus, Mycobacteriaceae.

Mycobacteria have characteristic rod-like shapes and waxy outer coats.

As such, Mycobacteria can be divided into three groups:
Mycobacterium tuberculosis complex—causative pathogen of tuberculosis
Mycobacterium leprae—causative pathogen of leprosy.
Nontuberculous mycobacteria (NTM) which encompass all other mycobacteria that are not M. tuberculosis or M. leprae, including Mycobacterium abscessus complex (MABSC), and Mycobacterium avium complex (MAC).

Tuberculosis (TB) is an infectious disease caused by Mycobacterium tuberculosis complex bacteria. As one of the oldest documented infectious agents in humans, TB remains a significant cause of mortality and morbidity worldwide, with an estimated causing 10.4 million new cases of TB infection, and 1.4 million people to die by active TB disease in 2015 (see, for example, World Health Organization (WHO) Global Tuberculosis Report 2016). In addition to the high prevalence and mortality rates, the incidence of multi-drug resistant tuberculosis (MDR-TB) is a growing concern, with 580,000 patients presenting with a drug-resistant TB infection in 2015. Co-morbidities, such as human immunodeficiency virus (HIV), complicate treatment, and were responsible for 1.2 million cases of TB in 2015.

To treat multi-drug resistant (MDR) infections, the WHO has recommended implementing a 9 to 12 month treatment regimen of second-line anti-TB drugs. These regimens, such as the 9 to 12 month Bangladesh regimen, treat MDR-TB with a combination of gatifloxacin, ethambutol, pyrazinamide, and clofazimine, which led to a relapse-free cure in 87.9% of patients (see, for example, Sotgiu, G, et al., “Applicability of the shorter ‘Bangladesh regimen’ in high multidrug-resistant tuberculosis settings”, International Journal of Infectious Diseases (2017) 56 190-193).

Other studies investigating shortened TB treatments demonstrated that clofazimine had no clinical benefit after two weeks of oral administration (see, for example, Diacon, A. H., et al., “Bactericidal Activity of Pyrazinamide and Clofazimine Alone and in Combinations with Pretomanid and Bedaquiline”, American Journal of Respiratory and Critical Care Medicine (2015), 191 (8), 943-953). The lack of activity was attributed to low bioavailability of the drug, as it was theorized to bind to circulating serum proteins with a high affinity. Despite the fact that clofazimine has been empirically demonstrated to be effective for the treatment of MDR-TB, and extensively-drug resistant TB (XDR-TB), its poor bioavailability after systemic administration appears to limit its biological activity over short duration therapies (see, for example, Swanson, R. V., et al., “Pharmacokinetics and Pharmacodynamics of Clofazimine in a Mouse Model of Tuberculosis”, Antimicrobial Agents and Chemotherapy (2015), 59 (6), 3042-3051).

Treatment of lung infections with inhaled antibiotics may result in higher drug concentrations in the lungs and reduced adverse effects compared to systemic delivery (see, for example, Touw, D. J., et al., “Inhalation of antibiotics in cystic fibrosis”, European Respiratory Journal (1995), 8, 1594-1604), which result in increased biological activity and efficacy (see, for example, Hickey, A. J., “Inhaled drug treatment for tuberculosis: Past progress and future prospects”, Journal of Controlled Release, (2016), 240, 127-134). In vivo mouse models have demonstrated that aerosolized administration of clofazimine shows significant improvement in bacilli clearance in TB-infection models compared to oral administration of clofazimine only 28 days after treatment initiation (see, for example, Verma, R. K., et al., “Inhaled microparticles containing clofazimine are efficacious in treatment of experimental Tuberculosis in Mice”, Antimicrobial Agents and Chemotherapy (2013), 57 (2), 1050-1052). This improved efficacy over a short duration is likely due to the direct delivery of clofazimine to the site of infection in the lungs resulting in higher clofazimine concentration in the pulmonary macrophages within the tuberculosis granulomas.

Accordingly, the use of an aerosolized administration of clofazimine in patients with MDR TB, or XDR-TB infections may further improve patient treatment outcomes and may shorten the duration of current treatment regimens.

The group of nontuberculous mycobacteria (NTM), formerly called atypical or ubiquitous mycobacteria, contains over 150 species. NTM can be found ubiquitously in nature and show a broad diversity. They can be detected in soil, ground and drinking water as well as in food like pasteurized milk or cheese. In general, NTM are considered to be less pathogenic. Nevertheless, they can cause severe illness in humans, especially in immune compromised persons or those who suffer from previous pulmonary diseases. Currently NTM are classified according to their growth rate and are divided into slow-growing (SGM) and rapid-growing (RGM) mycobacteria.

The slow growing Mycobacterium avium complex (MAC) comprises the species Mycobacterium avium, Mycobacterium chimaera and Mycobacterium intracellulare that are among the most important and most frequent pathogenic NTM. Just like Mycobacterium kansasii, Mycobacterium malmoense, Mycobacterium xenopi, simiae, Mycobacterium abscessus, Mycobacterium gordonae, Mycobacterium Mycobacterium fortuitum, and Mycobacterium chelonae, they mostly cause pulmonary infections. Mycobacterium marinum is responsible for skin and soft tissue infections like aquarium granuloma.

In particular, RGM cause serious, life-threatening chronic lung diseases and are responsible for disseminated and often fatal infections. Infections are typically caused by contaminated materials and invasive procedures involving catheters, non-sterile surgical procedures or injections and implantations of foreign bodies. Exposure to shower heads and jacuzzis has also been reported as risks for infections. NTM typically cause opportunistic infections in patients with chronic pulmonary diseases such as chronic obstructive pulmonary disease (COPD), cystic fibrosis (CF), and other immune compromised patients.

In recent years, the rapidly growing (RGM) Mycobacterium abscessus group strains (Mycobacterium abscessus complex, MABSC), comprising the subspecies Mycobacterium abscessus subsp. abscessus (M. a. abscessus), Mycobacterium abcessus bolletii and Mycobacterium abscessus massiliense have emerged as important human pathogens and are associated with significantly higher fatality rates than any other RGM.

Mycobacterium abscessus infection in CF patients is particularly problematic, as it results in enhanced pulmonary destruction and is often impossible to treat with failure rates as high as 60-66%. (see, for example, Obregon-Henao A et al, Antimicrobial Agents and Chemotherapy, November 2015, Vol 59, No 11, p. 6904-6912; Qvist, T., Pressler, T., Høiby, N. and Katzenstein, T L., “Shifting paradigms of nontuberculous mycobacteria in cystic fibrosis”, Respiratory Research (2014), 15(1):pp. 41-47). Human infection with NTM became of greater relevance with the emergence of the human acquired immune deficiency syndrome pandemic. Mycobacteria from Mycobacterium avium complex (MAC) were identified as the major cause of opportunistic infections in patients infected with the human immunodeficiency virus (HIV).

Several species of NTM are known to form biofilms. Biofilms are microcolonies of bacteria embedded in the extracellular matrix that provide stability and resistance to human immune mechanisms. In recent years, some species of NTM have been shown to form biofilms that enhance resistance to disinfectants and antimicrobial agents. Biofilm assembly proceeds through several phases, including reversible attachment, irreversible attachment, biofilm formation via bacterial aggregation, organization, and signaling, and finally dispersion. During this process, bacteria develop a matrix containing extracellular polymeric substances (EPS), such as polysaccharides, lipids and nucleic acids, to form a complex three-dimensional structure (see, for example, Sousa S. et al., International Journal of Mycobacteriology 4 (2015), 36-43). Specifically, mycobacterial EPS differ in nature from other biofilms, as mycobacteria do not produce exopolysaccharides (see, for example, Zambrano MM, Kolter R. Mycobacterial biofilms: a greasy way to hold it together. Cell. 2005). Mycobacterial biofilms vary between species, but can contain mycolic acids, glycopeptidolipids, mycolyl-diacylglycerols, lipooligosaccharides, lipopeptides, and extracellular DNA (Overview and original research from: Rose S J, Babrak L M, Bermudez L E (2015) Mycobacterium avium Possesses Extracellular DNA that Contributes to Biofilm Formation, Structural Integrity, and Tolerance to Antibiotics. PLoS ONE). The assembly in biofilms is known to enhance resistance to antimicrobial agents (see, for example, Faria S. et al., Journal of Pathogens, Vol 2015, Article ID 809014).

Delivery of aerosolized liposomal amikacin/inhaled amikacin solution nebulized by a jet nebulizer as a novel approach for treatment of NTM pulmonary infection has been suggested (Rose S. et al, 2014, PLOS ONE, Volume 9, Issue 9, e108703, and Olivier K. et al, Ann Am Thorac Soc Vol 11, No 1, pp. 30-35) as well as inhalation of anti-TB drugs dry powder microparticles for pulmonary delivery (Cholo M et al., J Antimicrob Chemother. 2012 February; 67(2):290-8 and Fourie B. and Nettey O., 2015 Inhalation Magazine, Verma 2013 Antimicrob Agents Chemother).

Multiple combination regimens with inhaled amikacin following initial treatment with parenteral aminoglycosides, tigecycline and other promising oral antibiotics such as linezolid, delamanid, and bedaquiline, and surgical intervention in selected cases have shown promising results in the treatment of NTM lung disease (Lu Ryu et al., Tuberc Respir Dis 2016; 79:74-84). However, the present inventors noted increasing incidence and prevalence of NTM infections, in particular NTM lung disease, and the limited treatment options necessitate the development of novel dosage forms/pharmaceutical formulations to enhance the bioavailability of the currently used oral antibiotics, which would provide better outcomes.

Combinations of oral clofazimine and amikacin have been shown to act synergistically in vitro against both Mycobacterium abscessus and Mycobacterium avium (see, for example, van Ingen, J., et al., “In Vitro Synergy between Clofazimine and Amikacin in Treatment of Nontuberculous Mycobacterial Disease”, Antimicrobial Agents and Chemotherapy 56 (12), 6324-6327 (2012)). Further, synergy has been shown with combinations of oral clofazimine and bedaquiline used against Mycobacterium tuberculosis (see, for example, Cokol, M. et al., “Efficient Measurement and factorization of high-order drug interactions in Mycobacterium tuberculosis”, Sciences Advances 2017:3:e170881, 11 Oct. 2017). Synergy has also been shown for a clofazimine/bedaquiline combination against the nontuberculous bacterium Myocbacterium abscessus (Ruth, M. M. et al., “A Bedaquiline/Clofazimine Combination Regimen Might Add Activity to the Treatment of Clinically Relevant Non-Tuberculous Mycobacteria”, Journal of Antimicrobial Chemotherapy (2019), doi.org/10.1093/jac/dky526).

Fungal pathogens have emerged as a leading cause of human mortality. Current estimates suggest death due to invasive fungal infections is on par with more well-known infectious diseases such as tuberculosis. Candida albicans, Cryptococus neoformans, and Aspergillis fumigatus represent the most prevalent fungal pathogens of humans. Each of these species is responsible for hundreds of thousands of infections annually with unacceptably high mortality rates due to poor diagnostics and limited treatment options. Clofazimine has been shown to exhibit efficacy as a combination agent against multiple fungi. (see, for example, Robbins, N., et al., “An Antifungal Combination Matrix Identifies a Rich Pool of Adjuvant Molecules that Enhance Drug Activity against Diverse Fungal Pathogens”, Cell Reports 13, 1481-1492 Nov. 17, 2015). Fungi also play a role as commensals, colonizers and/or pathogens in cystic fibrosis (see, for example, Chotirmall, S. H. and McElvaney, N. G., “Fungi in the cystic fibrosis lung: Bystanders or pathogens?”, The International Journal of Biochemistry & Cell Biology 52 (2014), 161-173.

The low solubility of clofazimine in water results in low oral bioavailability and high microbial resistance. The specific techniques required to solubilise and stabilize the drug in a pharmaceutic formulation in liquid aqueous carriers such as for aerosolization for nebulizers has been problematic.

New method of treatments are needed to combat NTM chronic infections. Accordingly, the inventors have developed new methods for the treatment of NTM in order to obtain deep lung deposition of the aerosol particles by enhancing the efficacy of the treatment and reducing adverse effects when compared to oral and parenteral therapies.

### SUMMARY

Disclosed herein are methods and compositions for the treatment of nontuberculous bacterial infection comprising, administering to a patient in need of treatment a pharmaceutical composition for inhalation comprising a therapeutically effective dose of a compound of the formula N,5-bis(4-chlorophenyl)-3-propan-2-yliminophenazin-2-amine, clofazimine, or an isolated form of a clofazimine isomer, a polymorphic form of clofazimine thereof selected from I, II, III, IV thereof, and/or combinations thereof, or a pharmaceutically acceptable salt thereof, wherein the clofazimine compound is provided in the form of a suspension, solution or dry powder; processes for their preparation; and uses and methods of treatment comprising them. Furthermore, the disclosure provides pharmaceutical compositions in a therapeutic combination with one or more than one active agent comprising, clofazimine in the form of an aerosol for pulmonary inhalation. For example, the disclosure provides a method for the treatment of an NTM infection with compositions for inhalation by nebulization, or by inhalation of a dry powder without serious adverse events to a patient being treated, including, no laboratory abnormalities, cardiac complications such as QT wave prolongation.

In one embodiment, the method of treatment comprises, administering to a patient in need of treatment an inhalable pharmaceutical composition comprising up to 10 mg, up to 30 mg, up to 60 mg, up to 90 mg, or up to about 100 mg of clofazimine compound, an isomer thereof, pharmaceutically acceptable salt, or polymorphic form thereof, which is administered to a patient once daily, wherein the patient inhales in one or more breaths for a period of a week, to about six months, or longer, which can be continuous for the entire period, or non-continuous, for example, having the patient take one or more rest periods for up to about a month interval between dosing regimens. In one embodiment, a patient is administered a pharmaceutical composition comprising clofazimine, or a polymormorphic form thereof in an amount of about 20 mg to about 100 mg, or from about 20 mg to about 90 mg, from about 25 mg to about 100 mg, from about 30 mg to about 90 mg for a period of six months, or longer depending on the patient's needs. In an embodiment, a patient is administered a pharmaceutical composition comprising clofazimine for a period of six months or longer, with intervals in-between treatment wherein the patient does not receive clofazimine treatment, for example, the resting period can be a period of about a week, two weeks, three weeks, a month or longer. The rest-periods can be determined for each individual patient with NTM depending on the severity of the disease or infection, and can comprise, from a few days and up to a month or longer. In one embodiment, the method can be a combination treatment, wherein the patient is administered an inhalable aerosolized clofazimine composition in combination with other drugs, for example, one or more than one drugs, including, steroid, antibiotics and the like. In this embodiment, the combinations and compositions provided herein may be used in the treatment and/or prophylaxis of pulmonary infections caused by mycobacteria and other gram-positive bacteria, and of pulmonary fungal infections. The combination treatment comprises the administration of an inhalable clofazimine composition with concomitantly or sequential administration of one or more drugs, which can be administered by inhalation or a different route, including, intravenous, subcutaneous, or oral administration. In this embodiment, the one or more drugs may include, antibiotics, including, amikacin, and other aminoglycosides with activity against NTM lung infection, including streptomycin, kanamycin, clarithromycin, pyrazinamide, rifampin, moxifloxacin, levofloxacin, and para-amino salicylate, and mixtures thereof, and the likes; anti-inflammatory drugs, including ibuprofen, prednisone, etc.

In an embodiment of the invention, a pharmaceutical composition is provided comprising:
(a) a therapeutically effective dose of clofazimine compound, a clofazimine isomer, a clofazimine polymorphic form, or a pharmaceutically acceptable derivative or salt thereof;
(b) a nonionic surfactant with a Hydrophilic-Lipophilic Balance value of greater than 10; for example, polysorbate 80, and
(c) an aqueous liquid carrier selected from water, isotonic saline, buffered saline and aqueous electrolyte solutions
wherein the clofazimine compound, isomer, polymorphic form, or the pharmaceutically acceptable derivative or salt thereof, is provided in the form of particles in a suspension,
and
wherein the particles of clofazimine, or the pharmaceutically acceptable derivative or salt thereof, have a median size of less than 5 μm and a D90 of less than 6 μm.

In another embodiment of the invention, the particles of clofazimine, or the pharmaceutically acceptable derivative or salt thereof, have a mean size of less than 2 μm and a D90 of less than 3 μm.

In another embodiment of the invention a pharmaceutical composition is provided comprising:
(a) a therapeutically effective dose of clofazimine;
(b) a nonionic surfactant with a Hydrophilic-Lipophilic Balance value of greater than 10; and
(c) an aqueous liquid carrier selected from water, isotonic saline, buffered saline and aqueous electrolyte solutions
wherein the clofazimine is provided in the form of particles in a suspension, and
wherein the particles of clofazimine have a median size of less than 5 μm and a D90 of less than 6 μm.

In another embodiment, the particles of clofazimine have a median size of less than 2 μm and a D90 of less than 3 μm. The present clofazimine compositions are stable at room temperature for up to about one year or more.

The aerosolization of the compositions of the invention by an appropriate nebulizer provides significantly increased delivery of the aerosolized clofazimine into the lower lung (i.e., to the bronchi, bronchioli, and alveoli of the central and lower peripheral lungs), thereby substantially enhancing the therapeutic efficacy.

The inhalation device should, moreover, preferably be further adapted for localized pulmonary delivery of an aerosol having an optimal particle size distribution for homogenous deposition in the lower lung.

The invention therefore provides for an aerosol having aerosol particles of sizes that facilitate delivery to the alveoli and bronchiole. A suitable aerodynamic particle size for targeting the alveoli and bronchiole is between 1 and 5 μm. Particles larger than that are selectively deposited in the upper lungs, namely bronchi and trachea and in the mouth and throat, i.e., oropharyngeal area. Accordingly, the inhalation device is configured as to produce an aerosol having a mass median aerodynamic diameter (MMAD) in the range from about 1 to about 5 μm, and preferably in the range from about 1 to about 3 μm. In a further embodiment, the particle size distribution is narrow and has a geometric standard deviation (GSD) of less than about 3.

Local lung delivery of the present clofazimine composition reduces the amount of compound that needs to be administered to a patient to obtain therapeutically effective dose, and thus, reduce the severe side effects, or toxicity generated by orally administered suspension, capsules or tablets. The reduce pulmonary administration of the clofazimine treatment herein to a patient in need is to local lung tissue and thus less toxic by decreasing the amount of the drug absorbed into the systemic circulation of a patient, which causes a range of side effects from inconvenient to life threatening events, most common of which are reversible upon cessation of treatment, including reduction in skin/conjunctival discoloration, ichthyosis, anorexia, diarrhea, corneal xerosis and enlargement of lymph nodes.

Also disclosed is an inhalable pharmaceutical composition comprising clofazimine, or a pharmaceutically acceptable derivative of clofazimine, a clofazimine salt, or a polymorph of clofazimine, or combination thereof, and a pharmaceutically acceptable carrier and/or excipient for use in the treatment or prophylaxis of a nontuberculous bacterial infection of the lungs, wherein clofazimine is in an amount of 1 mg to 20 mg wt % in the composition, and wherein the inhalable pharmaceutical composition is provided by inhalation in an effective daily dose of up to 90 mg of clofazimine. The i nontuberculous bacterial infection of the lungs treated or prophylactically dissuaded may be caused by a Mycobacterium selected from the group consisting of: Mycobacterium avium, Mycobacterium intracellulare, Mycobacterium abscessus, and Mycobacterium leprae, and a combination thereof. In one case, the nontuberculous bacterial infection is an opportunistic infection, selected from the group consisting of: Mycobacterium avium complex pulmonary disease and opportunistic nontuberculous infection, or combination thereof, concomitant with one or more of the group consisting of: cystic fibrosis, chronic obstructive pulmonary disease or acquired immune deficiency syndrome. Concomitant conditions may be treated such as the infection may be an opportunistic nontuberculous mycobacteria infection in a patient with cystic fibrosis. The inhalable pharmaceutical composition may also be used the infection treated or prophylactically dissuaded is caused by mycobacteria or other gram positive bacteria, and is administered by inhalation, before, simultaneously, or subsequent to the administration of an agent selected from the group consisting of: bedaquiline, or a pharmaceutically acceptable salt of derivative thereof, cefoxitine, amikacin, clarithromycin, pyrazinamide, rifampin, moxifloxacin, levofloxacin, and para-amino salicylate, and mixtures thereof.

In one instance, the clofazimine is at least about 90% orthorhombic polymorph III. The inhalable pharmaceutical composition may also be used to treat or as a prophylaxis against a nontuberculous bacterial infection of the lungs. The inhalable pharmaceutical composition may be delivered by inhalation for treatment or propylaxis with a high lung deposition rate of at least about 30%.

In yet another embodiment, there is disclosed an inhalable pharmaceutical composition comprising clofazimine, or a pharmaceutically acceptable derivative of clofazimine, a clofazimine salt, or a polymorph of clofazimine, or combination thereof, and a pharmaceutically acceptable carrier and/or excipient for use in the treatment or prophylaxis of a nontuberculous bacterial infection of the lungs, wherein clofazimine is in an amount of 1 mg to 20 mg wt % in the composition, and is delivered by an inhaler configured to cause high lung deposition rates of at least 30%.

### BRIEF DESCRIPTION OF THE DRAWINGS

FIGS. 1A and 1B depict Semi-log Plots of Mean±Standard Deviation Clofazimine Concentrations in Dog Plasma Following a Single Dose (Day 1) or 28 Consecutive Daily Doses (Day 28) of Low Dose (Group 3), Mid Dose (Group 4) or High Dose (Group 5) Clofazimine Inhalation Solution (CIS).

FIG. 2. Depicts a Log-log plot of dose dependence of plasma clofazimine pK in dog plasma. (Left panel-2A) Plasma Cmax was proportional to Dose 1.44 on SD 1 and 28. The Cmax on SD 28 was approximately 2.56 times that on SD 1. FIG. 2B (Right panel) AUC₀₋₂₄ was proportional to Dose1.47 on SD 1 and 28. AUC₀₋₂₄ on SD 28 was approximately 3.76 times that on SD 1.

FIG. 3 depicts a Semi-log plot of mean 6 standard deviation clofazimine concentrations in dog plasma for recovery period after 28 consecutive daily doses (day 28) of low-dose (group 3), mid-dose (group 4), or high-dose (group 5) CIS.

FIG. 4 depicts a graph illustrating of the study canines' lung and plasma levels of clofazimine postdosing at days 29, 56 and 84 of the study.

FIG. 5 depicts a graph illustrating the clofazimine concentrations in lung and plasma of canines on SD 29 for the low, middle and high mg/kg treatment.

FIG. 6 depicts a graph illustrating the clofazimine mean plasma concentration (semi-logarithmic scale) of human subjects treated with a single ascending dose of clofazimine at various times after administration of clofazimine for clofazimine doses of 30 mg, 60 mg, and 90 mg.

FIG. 7 depicts a graph illustrating the clofazimine mean plasma concentration (semi-logarithmic scale) of human subjects treated with a single ascending dose of clofazimine at various times after administration of clofazimine for clofazimine doses of 30 mg and 90 mg.

FIG. 8 depicts a graph illustrating the effects between surfactants used in experiments measuring cell survival. Three cell lines, A549 (dark-color bars), Calu-3 (light gray bars) and hAELVi (lighter gray bars) were tested for viability in medium containing a super-refined surfactant (PS 80) containing an oleic acid of about 70%. HBSS represents negative control and 1% Triton X-100 was used as positive control. data shown in the graphs.

FIG. 9 depicts a graph illustrating the effects between surfactants used in experiments measuring cell survival. Three cell lines, A549 (dark-color bars), Calu-3 (light gray bars) and hAELVi (lighter gray bars) were tested for viability in medium containing a super-refined surfactant (PS 80) containing an oleic acid of about 99%. HBSS represents negative control and 1% Triton X-100 was used as positive control. data shown in the graphs.

### DETAILED DESCRIPTION

The present disclosure describes the unexpected discovery of therapeutically effective pharmaceutical formulations for lung delivery were made in which clofazimine or isomers can be readily aerosolized. The formulations facilitate the delivery by pulmonary aerosol administration of clofazimine in the form of a suspension, and achieve lower (i.e., deeper) lung deposition to the alveolar capillaries of the active agent, thereby significantly increasing the bioavailability of the extremely hydrophobic BCS class II agent, which results in significantly increased therapeutic efficacy coupled with reduced systemic side effects. In a particularly efficacious embodiment in regard to anti-mycobacterial activity, non-tuberculosis bacterial activity, and anti-inflammatory activities, orthorhombic polymorph III of clofazimine, is used in substantially pure form of at least about 85% more preferably at least about 90%, more preferably at least about 95%, more preferably at least about 97%, and yet more preferably at least about 99%.

In another aspect, this finding leads to the provision of an improved antibiotic therapy for infections caused by mycobacteria and gram-positive bacteria, in particular of pulmonary infections with NTM, such as opportunistic infections in Cystic Fibrosis (CF), Chronic-obstructive pulmonary disease (COPD) and immune compromised patients, including, HIV patients.

The present disclosure provides a more effective therapeutic regimen that also aims at preventing/overcoming and/or reducing systemic side effects that are caused by established, oral treatment regimens for pulmonary infections with gram positive bacteria, in particular, TB and NTM infections of the lungs as well as at the reduction of dose and of duration of treatment with clofazimine needed to treat the infections.

It is understood by the person of skill in the art that the present application also discloses each and any combination of the individual features disclosed herein.

### Definitions

As used herein, unless specifically defined, the term “clofazimine” can include, a clofazimine compound, a clofazimine isomer, a clofazimine polymorph, including, polymorphic form I, II, III or IV, clofazimine derivative, clofazimine analog, or pharmaceutically acceptable salt thereof, and or combinations thereof.

The term “pharmaceutically acceptable salt” refers to salts that retain the biological effectiveness and properties of the compounds of this invention and, which are not biologically or otherwise undesirable. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobionic acid, tartaric acid, benzoic acid, cinnamic acid, mandelic acid, methanesulfonic acid, ethanesulfonic acid, p-toluenesulfonic acid, salicylic acid, and the like.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

In accordance herewith, apart from the free base, the use of the methanesulfonic acid, maleic acid, isonicotinic acid, nicotinic acid, malonic acid, and salicylic acid salts, and in particular of clofazimine mesylate is preferred.

By the term “pharmaceutically acceptable derivative” as used herein, for example, compounds disclosed in U.S. Pat. No. 9,540,336 are meant, the disclosure of U.S. Pat. No. 9,540,336 is incorporated herein in its entirety. In addition, derivatives are meant as described in Lu, Y., Zhen, M., Wang, B., Fu, L., Zhao, W., Li, P., Xu, J., Zhu, H., Jin, H., Yin, D., Huang, H., Upton, A M. and Ma, Z., “Clofazimine Analogs with Efficacy against experimental Tuberculosis and reduced Potential for Accumulation” Antimicrobial Agents and Chemotherapy (2011), 55(11):pp.5185-5193. Additionally, The term, “pharmaceutically acceptable derivative” of a compound is, for example, a prodrug of said compound. In general, a prodrug is a derivative of a compound which, upon administration, is capable of providing the active form of the compound. Such derivatives, for example, may be an ester or amide of a carboxyl group, a carboxyl ester of a hydroxyl group, or a phosphate ester of a hydroxyl group.

By “therapeutically effective amount”, “therapeutically effective dose”, or “pharmaceutically effective amount” is meant an amount of clofazimine, or a pharmaceutically acceptable salt or derivative thereof, as disclosed for this invention, which has a therapeutic effect. The doses of clofazimine which are useful in treatment are therapeutically effective amounts. Thus, as used herein, a therapeutically effective amount means those amounts of clofazimine which produce the desired therapeutic effect as judged by clinical trial results and/or model animal infection studies.

The amount of clofazimine composition and daily dose to be administered to a patient can be determined and will vary, depending on several factors, such as the particular microbial strain involved. The dosage amount can further depend upon the patient's height, weight, sex, age and medical history. For prophylactic treatments, a therapeutically effective amount is that amount which would be effective to prevent a microbial infection.

A “therapeutic effect” relieves, to some extent, one or more of the symptoms of the infection, and includes curing an infection. “Curing” means that the symptoms of active infection are eliminated, including the total or substantial elimination of excessive members of viable microbe of those involved in the infection to a point at or below the threshold of detection by traditional measurements. However, certain long-term or permanent effects of the infection may exist even after a cure is obtained (such as extensive tissue damage). As used herein, a “therapeutic effect” is defined as a statistically significant reduction in bacterial load in a host, emergence of resistance, or improvement in infection symptoms as measured by human clinical results or animal studies.

“Treat”, “treatment”, or “treating” as used herein refers to administering a pharmaceutical composition/combination for prophylactic and/or therapeutic purposes.

The term “prophylactic treatment” refers to treating a patient who is not yet infected, but who is susceptible to, or otherwise at risk of, a particular infection. The term “therapeutic treatment” refers to administering treatment to a patient already suffering from an infection. Thus, in preferred embodiments, treating is the administration to a mammal (either for therapeutic or prophylactic purposes) of therapeutically effective amounts of clofazimine.

Unless stated otherwise herein, the term “inhalation” is meant to refer to pulmonary inhalation.

Unless stated otherwise herein, the term “infection” as used herein is meant to refer to pulmonary infections.

Unless otherwise stated, the term “substantially” when used to refer to the purity of a compound, indicates a purity of compound of 95% or greater purity.

Unless otherwise stated, the term “appropriate particle size” refers to a particle size of clofazimine in a composition, or a composition that provides the desired therapeutic effect when administered to a patient.

Unless otherwise stated, the term “appropriate concentration” refers to a concentration of a component in a composition or combination which provides a pharmaceutically acceptable composition or combination.

### Pharmaceutical Compositions and Combinations

The following water grades are particularly applicable to the present invention: sterile purified water, sterile water for injection, sterile water for irrigation, sterile water for inhalation (USP) and corresponding water grades in accordance with e.g. European Pharmacopoeia or National Formulary.

Aqueous electrolyte solutions as used in accordance with the present invention as the aqueous liquid carrier may further comprise sodium chloride, potassium chloride, lithium chloride, magnesium chloride, calcium chloride or mixtures thereof. The aqueous liquid carrier is preferably isotonic saline solution (0.9% NaCl corresponding to about/approximately 150 mM NaCl, preferably 154 mM NaCl).

Clofazimine has been shown to exist in at least four polymorphic forms (see, for example, Bannigan, et al., “Investigation into the Solid and Solution Properties of Known and Novel Polymorphs of the Antimicrobial Molecule Clofazimine”, Cryst. Growth Des. 2016, 16 (12), pp. 7240-7250). Clofazimine can exist in a triclinic form FI, a monoclinic form FII, and an orthorhombic form FIII. A further form FIV has also been seen only at high temperatures.

Accordingly, in a further embodiment of the invention a pharmaceutical composition is provided comprising:
(a) a therapeutically effective dose of clofazimine;
(b) a nonionic surfactant with an Hydrophilic-Lipophilic Balance value of greater than 10; and
(c) an aqueous liquid carrier selected from water, isotonic saline, buffered saline and aqueous electrolyte solutions
wherein the clofazimine is provided in the form of particles in a suspension, and
wherein the particles of clofazimine have a median size of less than 5 μm and a D90 of less than 6 μm, preferably a median size of less than 2 μm and a D90 of less than 3 μm, and wherein the clofazimine is provided in a polymorphic form or forms selected from triclinic form FI, monoclinic form FII and orthorhombic form FIII and mixtures of such forms.

In another embodiment, the clofazimine is provided substantially in orthorhombic form FIII.

In a further embodiment, a pharmaceutical composition according to any of the composition embodiments herein described is provided wherein the nonionic surfactant is selected from polysorbate 20 (for example Tween® 20, polysorbate 60 (for example Tween® 60), polysorbate 80 (for example Tween® 80), stearyl alcohol, a polyethylene glycol derivative of hydrogenated castor oil with an Hydrophilic-Lipophilic Balance value of 14 to 16 (for example Cremophor® RH 40), a polyethylene glycol derivative of hydrogenated castor oil with an Hydrophilic-Lipophilic Balance value of 15 to 17 (for example Cremophor® RH 60), sorbitan monolaurate (for example Span® 20), sorbitan monopalmitate (for example Span® 40), sorbitan monostearate (for example Span® 60), polyoxyethylene (20) oleyl ether (for example Brij® 020), polyoxyethylene (20) cetyl ether (for example Brij® 58), polyoxyethylene (10) cetyl ether (for example Brij® C10), polyoxyethylene (10) oleyl ether (for example Brij® 010), polyoxyethylene (100) stearyl ether (for example Brij® S100), polyoxyethylene (10) stearyl ether (for example Brij® S10), polyoxyethylene (20) stearyl ether (for example Brij® S20), polyoxyethylene (4) lauryl ether (for example Brij® L4), polyoxyethylene (20) cetyl ether (for example Brij® 93), polyoxyethylene (2) cetyl ether (for example Brij® S2), caprylocaproyl polyoxyl-8 glyceride (for example Labrasol®), polyethylene glycol (20) stearate (for example Myrj™ 49), polyethylene glycol (40) stearate (for example Myrj™ S40), polyethylene glycol (100) stearate (for example Myrj™ S100), polyethylene glycol (8) stearate (for example Myrj™ S8), and polyoxyl 40 stearate (for example Myrj™ 52), and mixtures thereof.

In embodiments herewith, the composition for treating a lung infection comprises a pharmaceutical composition comprising, an active agent, including and antimicrobial such as clofazimine, and a surfactant, including, including, polysorbate 80; wherein the surfactant comprises one or more than one fatty acid, including, mysteric acid, palmitic acid, palmitoleic acid, stearic acid, oleic acid, linoleic acid, and linolenic acid, and/or combinations thereof. In some embodiments, the surfactant comprises palmitic acid in an amount of up to about 18 wt %; up to about 10 wt % palmitoleic acid; or up to about 6% stearic acid in the composition. In a particular embodiment, the surfactant comprises oleic acid in the composition in an amount of up to 100 wt %. In one embodiment, the surfactant in the composition comprises an oleic acid content ranging from about 70 wt % to about 99 wt %. In some embodiments, the surfactant comprises from about 80 wt % to about 99 wt %, or from about 83 wt % to about 95 wt %; or from about 85 wt % to about 93 wt % in the composition.

In one embodiment, the composition for treating a lung infection comprises an active agent, including clofazimine, saline solution, and a surfactant comprising a fatty content other than oleic acid is less than 10 wt % of the composition.

In an exemplary embodiment, a dose of a composition for treating lung disease, including a lung infection is provided comprising clofazimine in an amount of up to 150 mg in the composition; a saline solution of from about 1% to about 7%, and a surfactant comprising from about 80% to about 99.5% oleic acid.

In another embodiment, a pharmaceutical composition according to any of the composition embodiments described herein is provided, wherein the non-ionic surfactant is polysorbate 80, and wherein the aqueous liquid carrier is distilled water, hypertonic saline or isotonic saline. In another embodiment of the invention, a pharmaceutical composition is provided wherein the hypertonic saline is from 1% to 7% (w/v) sodium chloride. In a further embodiment of the invention, a pharmaceutical composition is provided wherein the non-ionic surfactant is polysorbate 80, and wherein the aqueous liquid carrier is isotonic saline.

In another embodiment, a pharmaceutical composition according to any one of the composition embodiments described herein is provided wherein the osmolality of the composition is in the range of 200-700 mOsm/kg. In a further embodiment, the osmolality of the composition is in the range of 300-400 mOsm/kg.

In a further embodiment, a pharmaceutical composition according to any one of the composition embodiments described herein, is provided wherein the nonionic surfactant is in the range of 0.001% to 5% (v/v), 0.05% to about 2.5%, or from about 0.01% to about 1% of the total composition and the amount of clofazimine is in the range of 0.1% to 30% (w/v) of the total composition.

In another embodiment of the invention, a pharmaceutical composition according to any one of the composition embodiments described herein is provided, wherein the pharmaceutical composition is prepared by a process comprising the following steps:
(1) homogenization of a suspension of clofazimine, the nonionic surfactant and water to obtain a suspension comprising clofazimine of an appropriate particle size,
(2) adjusting the pH of the suspension resulting from (1) to a pH of between pH 5.5 and pH 7.5, and
(3) adjusting the sodium chloride concentration to an appropriate concentration and
(4) adjusting the osmolality to an appropriate level.

In a further embodiment, the pH is adjusted to 7.4, and the sodium chloride concentration is adjusted to 154 mM sodium chloride. In another embodiment, the homogenization in step (1) is carried out by high pressure homogenization, high shear homogenization, wet milling, ultrasonic homogenization, or a combination of such processes. In another aspect, the homogenization of clofazimine is carried out in multiple steps of homogenization. In another embodiment, the appropriate particle size of the clofazimine are particles having a mean size of less than 5 μm and D90 of less than 6 μm. In a further embodiment, the appropriate particle size of clofazimine are particles having a mean size of less than 2 μm and D90 of less than 3 μm. In a further embodiment, a pharmaceutical composition according to any one of the composition embodiments described herein is provided, wherein the pharmaceutical composition is prepared by a process comprising the following steps:
(1) homogenization of a suspension of clofazimine and a non-aqueous liquid to obtain a suspension comprising clofazimine of an appropriate particle size,
(2) isolation of the clofazimine,
(3) addition of the clofazimine to the nonionic surfactant and water,
(4) adjusting the pH of the suspension resulting from (3) to a pH of between pH 5.5 and pH 7.5, and
(5) adjusting the sodium chloride concentration to an appropriate concentration.

In a further embodiment, the pH is adjusted to 7.4, and the sodium chloride concentration is adjusted to 154 mM sodium chloride. In a further embodiment, the homogenization in step (1) is carried out by high pressure homogenization, high shear homogenization, wet milling, ultrasonic homogenization, or a combination of such processes. In another embodiment, the homogenization of clofazimine is carried out in multiple steps of homogenization. In another embodiment, the appropriate particle size of the clofazimine are particles having a mean size of less than 5 μm and D90 of less than 6 μm. In a further embodiment, the appropriate particle size of clofazimine are particles having a mean size of less than 2 μm and D90 of less than 3 μm.

In a further embodiment, a pharmaceutical composition according to any one of the composition embodiments described herein is provided, wherein the composition is prepared by a process comprising the following steps:
(1) micronization of clofazimine to obtain clofazimine of an appropriate particle size,
(2) addition of the clofazimine to the nonionic surfactant and water,
(3) adjusting the pH of the suspension resulting from (2) to a pH of between pH 5.5 and pH 7.5, and
(4) adjusting the sodium chloride concentration to an appropriate concentration.

In a further embodiment, the pH is adjusted to 7.4, and the sodium chloride concentration is adjusted to 154 mM sodium chloride.

In another embodiment, the micronization of the clofazimine is carried out by jet milling, spray drying, ball milling, or super critical fluids processing. In another embodiment, the micronization of clofazimine is carried out in multiple steps of micronization. In another embodiment, the appropriate particle size of the clofazimine are particles having a mean size of less than 5 μm and D90 of less than 6 μm. In a further embodiment, the appropriate particle size of clofazimine are particles having a mean size of less than 2 μm and D90 of less than 3 μm.

In a further embodiment, a pharmaceutical composition according to any one of the composition embodiments described herein is provided, wherein the composition is prepared by a process comprising homogenization of a suspension of clofazimine in the nonionic surfactant, water containing an appropriate concentration of sodium chloride, and which has been adjusted to a pH of between pH 5.5 and pH 7.5, to obtain clofazimine of an appropriate particle size. In a further embodiment, the pH is adjusted to 7.4, and the sodium chloride concentration is adjusted to 154 mM sodium chloride. In a further embodiment, the homogenization is carried out by high pressure homogenization, high shear homogenization, wet milling, ultrasonic homogenization, or a combination of such processes. In another embodiment, the homogenization of clofazimine is carried out in multiple steps of homogenization. In another embodiment, the appropriate particle size of the clofazimine are particles having a mean size of less than 5 μm and D90 of less than 6 μm. In a further embodiment, the appropriate particle size of clofazimine are particles having a mean size of less than 2 μm and D90 of less than 3 μm.

In another embodiment, a process for the preparation of a pharmaceutical composition according to any of the composition embodiments described herein is provided, comprising the following steps:
(1) homogenization of a suspension of clofazimine, the non-ionic surfactant and water to obtain a suspension comprising clofazimine of an appropriate particle size,
(2) adjusting the pH of the suspension resulting from (1) to a pH of between pH 5.5 and pH 7.5, and
(3) adjusting the sodium chloride concentration to an appropriate concentration, and
(4) adjusting the osmolality to an appropriate level.

In another embodiment, the pH is adjusted to 7.4, and the sodium chloride concentration is adjusted to 154 mM sodium chloride. In a further embodiment, the homogenization is carried out by high pressure homogenization, high shear homogenization, wet milling, ultrasonic homogenization, or a combination of such processes. In a further embodiment, the homogenization of clofazimine is carried out in multiple steps of homogenization. In a further embodiment, the appropriate particle size of clofazimine are particles having a mean size of less than 5 μm and a D90 of less than 6 μm. In another embodiment, the appropriate particle size of clofazimine are particles having a mean size of 2 μm and a D90 of less than 3 μm.

In another embodiment, a process for the preparation of any of the pharmaceutical composition embodiments as described herein is provided, wherein,
(1) homogenization of a suspension of clofazimine and a non-aqueous liquid to obtain a suspension comprising clofazimine of the appropriate particle size,
(2) isolation of the clofazimine,
(3) addition of the clofazimine to the nonionic surfactant and water,
(4) adjusting the pH of the suspension resulting from (3) to a pH of between pH 5.5 and pH 7.5, and
(5) adjusting the sodium chloride concentration to an appropriate concentration.

In another embodiment, the pH is adjusted to 7.4, and the sodium chloride concentration is adjusted to 154 mM sodium chloride. In a further embodiment, the homogenization is carried out by high pressure homogenization, high shear homogenization, wet milling, ultrasonic homogenization, or a combination of such processes. In a further embodiment, the homogenization of clofazimine is carried out in multiple steps of homogenization. In a further embodiment, the appropriate particle size of clofazimine are particles having a mean size of less than 5 μm and a D90 of less than 6 μm. In another embodiment, the appropriate particle size of clofazimine are particles having a mean size of 2 μm and a D90 of less than 3 μm.

In a further embodiment, a process for the preparation of a pharmaceutical composition according to any one of the pharmaceutical composition embodiments as described herein is provided, comprising the following steps:
(1) micronization of clofazimine to obtain clofazimine of an appropriate particle size,
(2) addition of the clofazimine to the nonionic surfactant and water,
(3) adjusting the pH of the suspension resulting from (2) to a pH of between pH 5.5 and pH 7.5, and
(4) adjusting the sodium chloride concentration to an appropriate concentration.

In another embodiment, the pH is adjusted to 7.4, and the sodium chloride concentration is adjusted to 154 mM sodium chloride. In a further embodiment, the micronization of the clofazimine is carried out by jet milling, spray drying, ball milling, or super critical fluids processing. In a further embodiment, the micronization of clofazimine is carried out in multiple steps of micronization. In a further embodiment, the appropriate particle size of clofazimine are particles having a mean size of less than 5 μm and a D90 of less than 6 μm. In another embodiment, the appropriate particle size of clofazimine are particles having a mean size of 2 μm and a D90 of less than 3 μm.

In another embodiment, a process for the preparation of a pharmaceutical composition according to any one of pharmaceutical composition embodiment described herein is provided, comprising homogenization of a suspension of clofazimine in the nonionic surfactant, water containing an appropriate concentration of sodium chloride, and which has been adjusted to a pH of between pH 5.5 and pH 7.5, to obtain clofazimine of an appropriate particle size. In another embodiment, the pH is 7.4, and the appropriate concentration of sodium chloride is 154 mM sodium chloride. In a further embodiment, the homogenization is carried out by high pressure homogenization, high shear homogenization, wet milling, ultrasonic homogenization, or a combination of such processes. In a further embodiment, the homogenization of clofazimine is carried out in multiple steps of homogenization. In a further embodiment, the appropriate particle size of clofazimine are particles having a mean size of less than 5 μm and a D90 of less than 6 μm. In another embodiment, the appropriate particle size of clofazimine are particles having a mean size of 2 μm and a D90 of less than 3 μm.

In a further embodiment, a process for the preparation of a pharmaceutical composition according to any one of composition embodiments described herein, is provided, comprising the following steps: (a) homogenization of a suspension of clofazimine, the non-ionic surfactant and water to obtain a suspension comprising clofazimine of an appropriate particle size; (b) adjusting the pH of the resulting suspension a pH of between pH 5.5 and pH 7.5; (c) adjusting the sodium chloride concentration to an appropriate concentration, and (d) adjusting the osmolality to an appropriate level; and wherein steps (b), (c) and (d), may occur in the order of (b), (c), (d); (b), (d), (c); (c), (b), (d); (c), (d), (b); (d), (b), (c); or (d), (c), (b). In another embodiment, a process for the preparation of a pharmaceutical composition according to any one of the composition embodiments described herein, is provided comprising the following steps: (a) homogenization of a suspension of clofazimine and a non-aqueous liquid to obtain a suspension comprising clofazimine of the appropriate particle size; (b) isolation of the clofazimine; (c) addition of the clofazimine to the nonionic surfactant and water; (d) adjusting the pH of the resulting suspension to a pH of between pH 5.5 and pH 7.5; and (e) adjusting the sodium chloride concentration to an appropriate concentration; and wherein steps (d) and (e) may occur in the order of (d), (e); or (e), (d).

In another embodiment, a process for the preparation of a pharmaceutical composition according to any one or the composition embodiments described herein, is provided, comprising the following steps: (a) micronization of clofazimine to obtain clofazimine of an appropriate particle size, and (b) addition of the clofazimine to the nonionic surfactant, water containing an appropriate concentration of sodium chloride, and which has been adjusted to a pH of between pH 5.5 and 7.5.

In another embodiment, a pharmaceutical combination in the form of an aerosol for inhalation is provided, prepared by aerosolization of the composition according to any one of the composition embodiments described herein, by a nebulizing device selected from an ultrasonic nebulizer, an electron spray nebulizer, a vibrating membrane nebulizer, a jet nebulizer and a mechanical soft mist inhaler, and
wherein the aerosol particles produced by the nebulizing device have a mass median aerodynamic diameter of 1 to 5 μm. In a further embodiment, the aerosol for inhalation is for lower lung deposition. In another embodiment, the nebulizing device exhibits an output rate of 0.1 to 1.0 ml/min. In another embodiment, the total inhalation volume is between 1 ml and 5 ml.

In another embodiment, a pharmaceutical composition according to any one of the composition embodiments described herein is provided which is for use in combination with an agent for dispersing and/or destruction of biofilm, with mucolytic and/or mucoactive agents, and/or agents that reduce biofilm formation selected from nebulized 4-7% hypertonic saline, metaperiodate, sodium dodecyl sulfate, sodium bicarbonate, tromethamine, silver nano particles, bismuth thiols, ethylene diamine loaded phosphatidylcholine-decorated gold tetraacetic acid, gentamicin nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In another embodiment, the composition for the use is administered before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

In another embodiment, a pharmaceutical combination according to any of the combination embodiments described herein is provided which is for use in combination with an agent for dispersing and/or destruction of biofilm, with mucolytic and/or mucoactive agents, and/or agents that reduce biofilm formation selected from nebulized 4-7% hypertonic saline, metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In another embodiment, the combination for the use is used to administer a composition of the present invention before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the composition is administered before, simultaneously or subsequently to the administration of an agent selected from bedaquiline or a pharmaceutically acceptable salt or derivative thereof, and amikacin, and mixtures thereof. In a further embodiment, the composition is administered before, simultaneously or subsequently to the administration of bedaquiline or a pharmaceutically acceptable salt or derivative thereof.

In another embodiment, a pharmaceutical composition according to any one of the composition embodiments as described herein is provided for use in the treatment and/or prophylaxis of a pulmonary infection caused by mycobacteria or other gram positive bacteria. In a further embodiment, the infection is caused by a species of the genus Mycobacterium selected from nontuberculous mycobacteria and Mycobacterium tuberculosis complex, and a combination thereof. In a further embodiment, the nontuberculous mycobacteria is selected from Mycobacterium avium, Mycobacterium intracellulare, Mycobacterium abscessus, and Mycobacterium leprae, and a combination thereof. In another embodiment, the infection is an opportunistic infection, selected from MAC pulmonary disease and nontuberculous infection, in a patient with cystic fibrosis, chronic obstructive pulmonary or acquired immune deficiency syndrome. In another embodiment, the infection is an opportunistic nontuberculous mycobacteria infection in patients with cystic fibrosis. In another embodiment, the composition for the use is administered before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the composition is administered before, simultaneously or subsequently to the administration of an agent selected from bedaquiline or a pharmaceutically acceptable salt or derivative thereof, and amikacin, and mixtures thereof. In a further embodiment, the composition is administered before, simultaneously or subsequently to the administration of bedaquiline or a pharmaceutically acceptable salt or derivative thereof.

In another embodiment, a pharmaceutical combination according to any of the combination embodiments as described herein is provided for use in the treatment and/or prophylaxis of a pulmonary infection caused by mycobacteria or other gram positive bacteria. In a further embodiment, the infection is caused by a species of the genus Mycobacterium selected from nontuberculous mycobacteria and Mycobacterium tuberculosis complex, and a combination thereof. In a further embodiment, the nontuberculous mycobacteria is selected from Mycobacterium avium, Mycobacterium intracellulare, Mycobacterium abscessus, and Mycobacterium leprae, and a combination thereof. In another embodiment, the infection is an opportunistic infection, selected from MAC pulmonary disease and nontuberculous infection, in a patient with cystic fibrosis, chronic obstructive pulmonary or acquired immune deficiency syndrome. In another embodiment, the infection is an opportunistic nontuberculous mycobacteria infection in patients with cystic fibrosis. In another embodiment, the combination for the use is used to administer a composition of the present invention before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the combination for the use is used to administer a composition of the present invention before, simultaneously or subsequently to the administration of an agent selected from bedaquiline or a pharmaceutically acceptable salt or derivative thereof, and amikacin, and mixtures thereof. In another embodiment, the combination for the use is used to administer a composition of the present invention before, simultaneously or subsequently to the administration of bedaquiline or a pharmaceutically acceptable salt or derivative thereof.

In another embodiment, a system for use in providing antibiotic activity when treating or providing prophylaxis against a pulmonary infection caused by mycobacteria or other gram-positive bacteria is provided wherein the system comprises:
1) a nebulized pharmaceutical combination comprising:
   (a) a therapeutically effective dose of clofazimine;
   (b) a nonionic surfactant with a Hydrophilic-Lipophilic Balance value of greater than 10; and
   (c) an aqueous liquid carrier selected from water, isotonic saline, buffered saline and aqueous electrolyte solutions
and
2) a nebulizer,
   wherein the clofazimine is present in the form of a suspension,
   and
   wherein the aerosol particles produced by the system have a mass median aerodynamic diameter of 1 to 5 μm.

In a further embodiment, a pharmaceutical composition according to any one of composition embodiments described herein is provided, for use in the treatment and/or prophylaxis of pulmonary fungal infections or Clostridium difficile, or a combination thereof. In another embodiment, a pharmaceutical composition according to any one of composition embodiments described herein is provided, for use in the treatment and/or prophylaxis of pulmonary fungal infections. In a further embodiment, the pulmonary fungal infection is Candida albicans or Aspergilus fumigatus, or a combination thereof.

In a further embodiment, a pharmaceutical combination according to any one of the combination embodiments described herein is provided, for use in the treatment and/or prophylaxis of pulmonary fungal infections or Clostridium difficile, or a combination thereof. A pharmaceutical combination according to any one of combinations embodiments described herein is provided, for use in the treatment and/or prophylaxis of pulmonary fungal infections. In a further embodiment, the pulmonary fungal infection is Candida albicans or Aspergilus fumigatus, or a combination thereof.

In another embodiment, a method of treatment or prophylaxis of a pulmonary infection is provided, in a patient in need thereof, comprising administering by inhalation a composition according to any one the composition embodiments described herein. In another embodiment, the infection is caused by a species of the genus Mycobacterium selected from nontuberculous mycobacteria and Mycobacterium tuberculosis complex, and a combination thereof. In a further embodiment, the nontuberculous Mycobacterium is selected from Mycobacterium avium, Mycobacterium intracellulare, Mycobacterium abscessus, and Mycobacterium leprae, and a combination thereof. In a further embodiment, the infection is an opportunistic infection, selected from MAC pulmonary disease and nontuberculous infection, in a patient with cystic fibrosis, chronic obstructive pulmonary disease or acquired immune deficiency syndrome. In another embodiment, the infection is an opportunistic nontuberculous mycobacteria infection in a patient with cystic fibrosis. In a further embodiment, a method of treatment or prophylaxis of a pulmonary infection is provided caused by mycobacteria or other gram positive bacteria, in a patient in need thereof, comprising administering by inhalation a composition according to any one of the composition embodiments described herein, before, simultaneously, or subsequently to the administration of an agent selected from bedaquiline, or a pharmaceutically acceptable salt of derivative thereof, cefoxitine, amikacin, clarithromycin, pyrazinamide, rifampin, moxifloxacin, levofloxacin, and para-amino salicylate, and mixtures thereof. In another embodiment, the agent is bedaquiline or amikacin. In a further embodiment, the agent is bedaquiline.

### Particle Size and Distribution

The therapeutic effect of aerosolized therapies is dependent upon the dose deposited and its distribution. Aerosol particle size is one of the important variables in defining the dose deposited and the distribution of drug aerosol in the lung.

Generally, inhaled aerosol particles are subject to deposition by one of two mechanisms: impaction, which usually predominates for larger aerosol particles, and sedimentation, which is prevalent for smaller aerosol particles. Impaction occurs when the momentum of an inhaled aerosol particle is large enough that the particle does not follow the air stream and encounters a physiological surface. In contrast, sedimentation occurs primarily in the lower lung when very small aerosol particles which have traveled with the inhaled air stream encounter physiological surfaces as a result of gravitational settling.

Pulmonary drug delivery may be accomplished by inhalation of an aerosol through the mouth and throat. Aerosol particles having an aerodynamic diameter of greater than about 5 μm generally do not reach the lung; instead, they tend to impact the back of the throat and are swallowed and possibly orally absorbed. Aerosol particles having diameters of about 3 to about 5 μm are small enough to reach the upper-to mid-pulmonary region (conducting airways), but they are too large to reach the alveoli. Smaller aerosol particles, i.e., about 0.5 to about 3 μm, are capable of reaching the alveolar region. Aerosol particles having diameters smaller than about 0.5 μm tend to be exhaled during tidal breathing, but can also be deposited in the alveolar region by a breath hold.

Aerosols used in pulmonary drug delivery are made up of a wide range of aerosol particle sizes, so statistical descriptors are used. Aerosols used in pulmonary drug delivery are typically described by their mass median diameter (MMD), that is, half of the mass is contained in aerosol particles larger than the MMD, and half the mass is contained in aerosol particles smaller than the MMD. For particles with uniform density, the volume median diameter (VMD) can be used interchangeably with the MMD. Determinations of the VMD and MMD are made by laser diffraction. The width of the distribution is described by the geometric standard deviation (GSD). However, the deposition of an aerosol particle in the respiratory tract is more accurately described by the particle's aerodynamic diameter, thus, the mass median aerodynamic diameter is typically used. MMAD determinations are made by inertial impaction or time of flight measurements. For aqueous particles, VMD, MMD and MMAD should be the same. However, if humidity is not controlled as the aerosol transits the impactor, MMAD determinations will be smaller than MMD and VMD due to dehydration. For the purposes of this description, VMD, MMD and MMAD measurements are considered to be under controlled conditions such that descriptions of VMD, MMD and MMAD will be comparable.

Nonetheless, for the purpose of the description, the aerosol particle size of the aerosol particles will be given as MMAD as determined by measurement at room temperature with a Next Generation Impactor (NGI) in accordance with US Pharmacopeial Convention. In Process Revision <601> Aerosols, Nasal Sprays, Metered-Dose Inhalers, and Dry Powder Inhalers, Pharmacopeial Forum (2003), Volume Number 29, pages 1176-1210 also disclosed in Jolyon Mitchell, Mark Nagel “Particle Size Analysis of Aerosols from Medicinal Inhalers”, KONA Powder and Particle Journal (2004), Volume 22, pages 32-65.

In accordance with the present invention, the particle size of the aerosol is optimized to maximize the deposition of clofazimine at the site of infection and to maximize tolerability. Aerosol particle size may be expressed in terms of the mass median aerodynamic diameter (MMAD). Large particles (e.g., MMAD>5 μm) tend to deposit in the extrathoracic and upper airways because they are too large to navigate bends in the airways. Intolerability (e.g., cough and bronchospasm) may occur from upper airway deposition of large particles.

Thus, in accordance with a preferred embodiment, the MMAD of the aerosol should be less than about 5 μm, preferably between about 1 and 5 μm, more preferably below 3 μm (<3 μm).

However, a guided breathing maneuver can be used to allow larger particles to pass through the extrathoracic and upper airways and deeper into the lungs than during tidal breathing which will increase the central and lower lung deposition of the aerosol. A guided breathing maneuver may be as slow as 100 ml/min. Thus, when used with a guided breathing maneuver, the preferred MMAD of the aerosol should be less than about 10 μm.

Another equally important factor (in addition to aerosol particle size) is the particle size and size distribution of the solid particles, in this case clofazimine particle size and distribution. The size of a solid particle in a given aerosol particle must be smaller than the aerosol particle in which it is contained. A larger aerosol particle may contain one or more solid particles. Further, when dealing with dilute suspensions, a majority of aerosol particles may contain no solid particle.

Because of this, it is desirable to have solid drug particles that are significantly smaller than the MMAD of the aerosol particles.

Another consideration, for example when using a vibrating mesh nebulizer, the formulation is pumped through orifices in a plate, which breaks up the suspension into droplets. It follows, then, that the solid particles must also be smaller than these orifices, in order to pass through.

Solid particle size in the suspension may be given by the mean size of the particles, and also by the distribution of the particles. D90 values indicate that 90% of the particles within the suspension are of the mean size or smaller.

Nebulizer: For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Healthcare, Inc. and DeVilbiss Health Care, Inc. Vibrating mesh nebulizers rely upon either piezoelectric or mechanical pulses to respirable liquid droplets generate. Other examples of nebulizers for use with clofazimine described herein are described in U.S. Pat. Nos. 4,268,460; 4,253,468; 4,046,146; 3,826,255; 4,649,911; 4,510,929; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303, all of which are hereby incorporated by reference in their entirety. Commercial examples of nebulizers that can be used with the clofazimine compositions described herein include Respirgard II®, Aeroneb® Aeroneb® Pro, and Aeroneb® Go produced by Aerogen; AERx® and AERx Essence™ produced by Aradigm; Porta-Neb®, Freeway Freedom™, Sidestream, Ventstream and I-neb produced by Respironics, Inc.; and PARI LCPlus®, PARI LC-Star®, and e-Flow7m produced by PARI, GmbH. Further non-limiting examples are disclosed in U.S. Pat. No. 6,196,219.

In an embodiment, the pharmaceutical composition may be preferably aerosolized using a nebulising device selected from an ultrasonic nebulizer, an electron spray nebulizer, a vibrating membrane nebulizer, a jet nebulizer or a mechanical soft mist inhaler. It is preferred that the device controls the patient's inhalation flow rate either by an electrical or mechanical process. In a further preferred embodiment, the aerosol production by the device is triggered by the patient's inhalation, such as with an AKITA device.

Preferred (commercially available) examples of the above nebulizers/devices to be used in accordance with the present invention are Vectura fox, Pari eFlow, Pari Trek S, Philips Innospire mini, Philips InnoSpire Go, Medspray device, Aeroneb Go, Aerogen Ultra, Respironics Aeroneb, Akita, Medspray Ecomyst and Respimat.

Use in Treatment and/or Prophylaxis

The pharmaceutical compositions and pharmaceutical combinations (aerosols, aerosolized formulations) and systems according to the present invention are intended for the use in the treatment and/or prophylaxis of pulmonary infections caused by mycobacteria or other clofazimine susceptible bacteria, such as Staphylococcus aureus (including methicillin-resistant and vancomycin intermediate-resistant strains), Streptococcus pneumoniae, and Enterococcus spp. The pharmaceutical compositions and pharmaceutical combinations of the present invention may also be used for the treatment and/or prophylaxis of pulmonary fungal infections.

### Dosing of Clofazimine

In one embodiment, the pharmaceutical composition is delivered by nebulization in about 1-5 ml, preferably 1-2 ml of the pharmaceutical composition of the invention. Thus, the target fill dose is about 1-5 ml corresponding to 20-100 mg clofazimine, based on a clofazimine concentration in the pharmaceutical composition of about 20 mg/ml.

The daily lung dose (i.e. the dose deposited in the lung) of clofazimine to be administered in accordance with the present invention is about 5-10 mg, which corresponds to a nominal dose of 15-30 mg (device dose) in the case of M. abscessus infections.

It is understood that the person of skill in the art will routinely adjust the lung dose of clofazimine to be administered (and thus the fill/nominal dose/the volume to be nebulized) based on the minimum inhibitory concentration (MIC) of clofazimine for the respective bacteria strain well established in the art.

Depending on the dosing frequency, once or twice per day, the daily lung dose will be split accordingly.

In one embodiment, clofazimine is to be administered once or twice daily with a resulting total daily lung dose of about 5 to 10 mg.

### Mucolytic Agents/Biofilm Modifying Agents

In order to reduce sputum viscosity during aerosol treatment and to destroy existing biofilm, the treatment and/or prophylaxis according with the present invention can involve additional administration of mucolytic and/or biofilm destructing agents. These agents can be prepared in fixed combination or be administered simultaneously or subsequently to the pharmaceutical composition/aerosol combination comprising clofazimine in accordance with the present invention.

Agents for dispersing/destruction of the biofilm, mucolytic and/or mucoactive agents and/or agents that reduce biofilm formation to be used in accordance with the present invention are selected from nebulized 4-7% hypertonic saline, metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides.

Furthermore, also other pharmaceutically active agents may be used in combination with the pharmaceutical compositions/aerosol combinations in accordance with the present invention. Such active agents may be selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

These agents can be prepared in fixed combination or be administered prior to, simultaneously or subsequently to the pharmaceutical composition/aerosol combination comprising clofazimine in accordance with the present invention.

### EXAMPLES

The following examples serve to more fully describe the manner of using the above-described invention, as well as to set forth the best modes contemplated for carrying out various aspects of the invention. The Examples according to the invention are those falling within the scope of the claims herein. The exemplary compositions and combinations below have been prepared in accordance with the processes described herein.

### Example 1

Preparation of clofazimine compositions: 200 mg of clofazimine (as triclinic form 1), 90 mg of sodium chloride, and 9.5 ml of water were mixed with a high shear homogenizer two times at 10,000 rpm for 5 minutes. 0.5 ml of polysorbate 80 (NOF Hx2) was added. This mixture was treated with an ultrasonic probe seven times, 3 minutes each, with an amplitude of 70%. The volume was adjusted to 10 ml with water. This suspension was filtered through filter paper, to give the Composition of Example 1. The composition of Example 1 had a median particle size of clofazimine of 3.9 μm, with a D90 of 6.7 μm. The concentration of clofazimine was determined by ultraviolet/visible spectroscopy at 280 nm, calibrating with a stock solution of 1 mg/ml of clofazimine diluted in the mobile phase, and determined to be 7.16 mg/ml.

The composition of Example 1 is shown in Table 1:

TABLE 1

| | | |
|---|---|---|
| Clofazimine Form FI | 71.6 | mg |
| Polysorbate 80 (NOF Hx2) | 0.5 | ml |
| Sodium chloride | 90.0 | mg |
| Water (V/V) | 9.5 | ml |

### Preparation of Clofazimine of Orthorhombic Form III:

A slurry of clofazimine (10 g) in toluene (20 ml) was stirred at 40° C. in an oil bath for 72 hours using a magnetic stirrer at 800 rpm. The solid portion of the slurry was collected by filtration through a crucible and dried at a maximum temperature of 40° C. under vacuum in an oven. This yielded 8.64 g of clofazimine as substantially pure (>98%) orthorhombic form III.

### Example 2

Preparation of clofazimine suspension: A suspension containing 6 g of clofazimine of orthorhombic form III in 100 ml of water containing 0.5% polysorbate 80 and 0.6% sodium chloride homogenized for approximately 40 seconds at 10,000 rpm using a high shear mixer. The pre-formulation was prepared by adding 0.6% sodium chloride in water to give a volume of 300 ml. 300 ml of this suspension was wet milled, using a microfluidizer, for 15 minutes by circulation of the suspension at 5,000 psi. The suspension was further homogenized for 23 minutes at 25,000 psi. Particle size analysis was performed with a HORIBA LA 950 indicating a median particle size of 0.83 μm with a D90 value of about 1.2 μm. A concentration of clofazimine of 16.05 mg/ml was determined by ultraviolet/visible spectroscopy at 280 nm, calibrating with a stock solution of 1 mg/ml of clofazimine diluted in the mobile phase.

The composition of Example 2 is shown in Table 2

TABLE 2
Composition of Example 2

| | | |
|---|---|---|
| Clofazimine form III | 4.81 | g (16.05 mg/ml) |
| Polysorbate 80 (NOF Hx2) | 1.5 | ml (0.5% v/v) |
| Sodium Chloride | 1.8 | g (0.6% w/v) |
| Water | 298.5 | ml |

### Example 3

A suspension of clofazimine (crystal modification orthorhombic Form III) in a solution of water, sodium chloride and Polysorbate 80, was treated using a Microfluidizer® (Microfluidics, Westwood, MA, USA) Processor operated for 30 minutes at a pressure of 28,250 psi, to produce the Composition of Example 3, with the resulting particles of clofazimine having a median particle size of 1.28 μm and a D90 below 2 μm.

The composition of Example 3 is shown in Table 3.

TABLE 3
Composition of Example 3

| | | |
|---|---|---|
| Clofazimine (Form FIII) | 20 | mg/ml |
| Polysorbate 80 | 0.5% | (v/v) |
| Sodium Chloride | 0.9% | (w/v) |
| Water | 99.5% | (v/v) |

### Viscosity Measurements:

The viscosity of the Composition of Example 3 was tested using a STRESSTECH Rheometer in stress control mode. A double gap geometry was utilized, and the spindle was continuously rotated to ensure the particulates remained in suspension during temperature points. Viscosity was measured across 0.01, 0.05, and 0.1 Pa stress each at 20° C., 25° C., and 30° C. Two separate loadings were performed to obtain the average viscosities shown in Table 4 below.

TABLE 4
Viscosity Measurements

| Temperature (° C.) | Viscosity Pa · s |
|---|---|
| 20 | 1.146E−03 |
| 25 | 1.049E−03 |
| 30 | 9.831E−04 |

### Animal Models and Efficacy Testing:

Compositions of the present invention have been tested for their ability to inhibit growth of clinical NTM species in an acute in vivo pulmonary infection mouse model to obtain preliminary data to establish clofazimine concentration levels in lung tissue after direct respiratory delivery as opposed to systemic administration. Two separate mouse models are used in order to investigate pulmonary NTM infection, dependent on the bacterial species of interest. For testing, Mycobacterium avium 2285, and Mycobacterium abscessus 103 bacterial strains have been used (Strain details can be found in “Phylogenetic analysis of Mycobacterial species using whole genome sequences”. Hazbon M. H., Riojas M. A., Damon A. M., Alalade R., Cantwell B. J., Monaco A., King S., Sohrabi A. Submitted (SEP-2014) to the EMBL/GenBank/DDBJ databases). These two species have been previously used in literature as models of NTM infection (Obregon-Henao et al. 2015 Antimicrob Agents Chemother; and Chan et al. Animal Models of Non-Tuberculous Mycobacterial Infections, Mycobact Dis 2016).

### In Vivo Safety Study in Balb/C Mice:

For in vivo safety and tolerability, 6-8 week old Balb/C female mice are obtained from Charles River. The mice are rested for one week before dosing. For each dose of clofazimine, three healthy mice are given a total of three doses every other day. Mice were dosed at 10.0, 5.01, and 2.51 mg/kg of clofazimine in the composition of Example 1. The compounds were given to 3 healthy mice for a total of three doses, every other day, by Microsprayer® aerosol intratracheal administration.

Clofazimine was found to be safe at 20 mg/kg (gavage, 200 μl). The composition of Example 1 showed no toxicity at the highest dose tested (10.0 mg/kg; 0.2506 mg/dose in 35 μl intratracheally). Accordingly, the composition of Formula I was considered safe and well tolerated at 10.0 mg/kg.

### Determination of Minimum Inhibitory Concentration

Minimum inhibitory concentration (MIC) testing was performed by microbroth dilution method using Mueller Hinton (MH) broth (Cation Adjusted) to the calcium and magnesium ion concentration recommended in the CLSI standard M7-A7 (Becton Dickinson). MIC testing also was performed by microbroth dilution method using 7H9 broth (Sigma-Aldrich). The justification for use of both MH and 7H9 broth for compound screening is that antimycobacterial compounds have been shown to display different MIC activity depending on the broth that is used in the MIC assay. M. abscessus was grown on 7H11 agar plates (Sigma-Aldrich) for 3 days at 35-37° C. in ambient air (depending on bacterial strain), and M. avium was grown on agar 7H11 plates (Sigma-Aldrich) for 21-30 days at 37° C. in ambient air.

The colony forming units (CFUs) are taken from the agar plates and placed in either MH or 7H9 broth with 0.05% tween-80 and grown at 35-37° C. in ambient air until the optical density (OD) absorbance taken after 3 days (M. abscessus) or 12 (M. avium) of growth is an (OD) 0.08-0.1 (0.5 McFarland Standard). The bacterial cell suspensions are then confirmed by preparing them in saline, matching the (OD) 0.08-0.1 (0.5 McFarland Standard). Compound stock solutions were made by suspending the compounds in DMSO at a concentration of 1.28 mg/ml, and used immediately for test range 64-0.062 μg/ml. Following this, 180 μl of broth (either MH or 7H9) was added to the first column in the 96 well plates, and 100 μl of broth to the remaining columns in the 96 well plate. 20 μl of compound stock solution was added to the first column of wells, and serially diluted. Finally, 100 μl NTM cell suspension was added in all the wells except the media only control wells. QC agents specific for each organism 1) bacteria only negative control 2) media only negative control 3) clarithromycin positive drug controls.

M. abscessus ODs were assayed on day 3, and M. avium on day 12. Following these measurements, the plate was assayed by using the Resazurin Microtiter Assay Plate method. Briefly, the method uses the addition of resazurin (7-Hydroxy-3H-phenoxazin-3-one 10-oxide) to the 96 well plate. Resazurin is a blue dye, itself weakly fluorescent until it is irreversibly reduced to the pink colored and highly red fluorescent resorufin. It is used as an oxidation-reduction indicator to determine bacterial cell viability in MIC assays.

Assays were done in triplicate. Assay #1 was performed after storage of the Composition of Example 1 at 4° C. for 2 months, Assay #2 was performed at 4 months, and Assay #3 at five months.

### Minimum Inhibitory Concentrations in the Presence and Absence of CF Sputum

Minimum inhibitory concentrations assays were performed as described above. To investigate the effect of cystic fibrosis (CF) patient sputum on antimicrobial activity of clofazimine (CFZ) and the Composition of Example 1, sputum was collected from patients who had not received antibiotics for the previous 48 hours, and their sputum was sterilized by exposure to UV light to eliminate endogenous bacteria. Following sterilization, M. abscessus, M. avium, M. intracellulare, and M. Chimaera were incubated in 10% CF sputum before undergoing MIC testing. The MIC of the Composition of Example 1 was measured following the same CLSI protocol as described above, in the presence and absence of cystic fibrosis patient sputum. All studies were performed in duplicate.

MIC values of clofazimine and the Composition of Example 1 in the presence and absence of sputum are shown in Table 5.

TABLE 5

| | | | Composition | CFZ + | Composition |
|---|---|---|---|---|---|
| | Identification | CFZ | of Ex. 1 | SP | of Ex. 1 + SP |
| Species | Number | (μg/ml) | (μg/ml) | (μg/ml) | (μg/ml) |
| M. avium | ATCC 700898 | 0.25 | 0.125 | >2 | >2 |
| M. intracellulare | DSM 43223 | 1 | 0.125 | >2 | >2 |
| M. Chimaera | CIP 107892 | 0.5 | 0.125 | >2 | >2 |
| M. avium | B18101968 | 0.5 | 0.125 | >2 | >2 |
| M. abscessus | CIP104536 | 0.25 | 0.125 | >2 | >2 |
| M. abscessus | B18104072 | 1 | 0.25 | >2 | >2 |
| M. abscessus | B15029863 | 2 | 2 | >2 | >2 |
| M. abscessus | B12052284 | 2 | 0.5 | >2 | >2 |

The results presented in Table 5 indicate consistent MICs of both clofazimine and the Composition of Example 1, against a range of nontuberculous mycobacterial species.

These data indicate that the Composition of Example 1 demonstrates potent in vitro activity against both M. abscessus and M. avium, and is stable at least over this time period.

Mouse model of M. abscessus in the SCID mouse

6-8 week old SCID female mice were ordered from Charles River. Mice were rested one week before infection.

Working stocks of M. abscessus strain 103 were frozen in 1 ml aliquots and sotred at −80° C. before use. For infection an aliquot was thawed, disrupted 20 times with a 1 ml luer-lok syringe fitted with a 26 g needle, and diluted in sterile 1×PBS.

The acute SCID mouse model received a non-invasive intratracheal instillation pulmonary infection with 1×10⁶ CFU/mouse (M. abscessus strain 103).

Three mice were sacrificed day 1 post-infection to determine bacterial uptake. Whole lungs, spleens, and livers are extracted, homogenized in 4.5 ml of 1×PBS. Homogenates were serially diluted in 1:10 dilutions and dilutions (0-1-2-3-4-5-6-7) plated on 7H11 agar plates. The plates are placed in 32° C. dry-air incubator (strain dependent) for 7 days.

The Composition of Example 1 10.0 mg/kg was administered by a Microsprayer® (35 μl) through the pulmonary route, and clofazimine (gavage), amikacin (subcutaneous) in a volume of 200 μl per mouse which begins day 2 post-infection and continued every other day for 8 consecutive days.

Mice were sacrificed 2 days after administration of the last dose of the compounds. Six mice of all groups (untreated control, clofazimine (gavage), composition of Example 1, and amikacin treated mice) were sacrificed and bacterial loads were determined. Plating of lung homogenate at 0-1-2-3-4-5-6-7, spleen at 0-1-2-3-4-5-6-7 and liver at 0-1-2-3-4-5-6-7.

Log 10 protection values of at least 0.60 indicate activity is statistically significant. Statistical analysis was performed by first converting CFU to logarithms, which were then evaluated by a one-way ANOVA followed by a multiple comparison analysis of variance by a one-way Tukey test (GraphPad Prism analysis software). Differences are considered significant at the 95% level of confidence.

Table 6 shows the average Log₁₀ CFU data and standard error of mean (SEM) following SCID mouse M. abscessus infection, where “n” is the total number of animals in group at time of sacrifice.

TABLE 6

| | Lung | Spleen | Liver |
|---|---|---|---|
| | Log₁₀ CFU ± | Log₁₀ CFU ± | Log₁₀ CFU ± |
| Group | SEM | SEM | SEM |
| Day 1 (Pre-treatment Control) (n = 3) | 5.08 ± 0.03 | 4.42 ± 0.04 | 5.00 ± 0.11 |
| Treatment End | | | |
| Saline Control (IT) (n = 6) | 5.51 ± 0.06 | 6.33 ± 0.85 | 6.45 ± 0.34 |
| Amikacin (SQ) 150 mg/kg (n = 6) | 3.94 ± 0.11 | 4.37 ± 0.31 | 3.07 ± 0.10 |
| Clofazimine (Oral) 20 mg/kg (n = 6) | 3.33 ± 0.17 | 3.79 ± 0.10 | 4.78 ± 0.18 |
| Composition of Example 1 (IT, | 2.66 ± 0.28 | 3.29 ± 0.25 | 4.57 ± 0.14 |
| aerosol) 10.0 mg/kg (n = 6) | | | |

The data in Table 6 indicate that treatment with the composition of Example 1 led to the greatest reduction in bacterial recovery in the lungs and spleen of animals infected with M. abscessus. This bacterial reduction was statistically improved over treatment with amikacin, or oral clofazimine.

Mouse Model of M. avium Infection in the Beige Mouse

In this study, 6-8 week old Beige female mice were ordered from Charles River. Mice were rested one week before infection.

The acute Beige mouse model received a non-invasive aerosol exposure pulmonary infection with 1×10⁸ colony forming units (CFU)/ml (M. avium strain 2285 rough). Working stocks of M. avium strain 2285 rough were frozen in 1 ml aliquots and stored at −80° C. before use. For infection an aliquot was thawed, disrupted 20 times with a 1 ml luer-lok syringe fitted with a 26 g needle, and diluted in sterile 1× phosphate buffered saline (PBS).

Three mice were sacrificed on day 1 and day 7 post-infection to determine bacterial uptake. Whole lungs, spleens, and livers were extracted, homogenized in 4.5 ml of 1×PBS and diluted 1:10. Dilutions (0-1-2-3-4-5-6-7) are plated on 7H11/OADC, TSA and charcoal agar plates and incubated at 32° C. in a dry-air incubator (strain dependent) for 30 days.

The composition of Example 1, 10.0 mg/kg was administered by a Microsprayer® (35 μl) though the pulmonary route and clofazimine (gavage) in a volume of 200 μl per mouse which begins on day 7 post-infection and continued every other day for 10 consecutive days.

Mice were sacrificed 5 days after administration of the last dose of the compounds. Six mice of all groups (untreated control, clofazimine (gavage), and the composition of Example 1) were sacrificed and bacterial loads were determined. Plating of lung homogenate at 0-1-2-3-4-5-6-7, spleen at 0-1-2-3-4-5-6-7 and liver at 0-1-2-3-4-5-6-7.

Log 10 protection values of at least 0.60 indicate activity is statistically significant. Statistical analysis was performed by first converting CFU to logarithms, which were then evaluated by a one-way ANOVA followed by a multiple comparison analysis of variance by a one-way Tukey test (SigmaStat software program). Differences are considered significant at the 95% level of confidence.

Table 7 shows the average Log₁₀ CFU data following Beige mouse M. avium infection.

TABLE 7

| | Lung | Spleen | Liver |
|---|---|---|---|
| | Log₁₀ | Log₁₀ | Log₁₀ |
| | CFU ± | CFU ± | CFU ± |
| Group | SEM | SEM | SEM |
| Day 1 (Pre-treatment Control) (n = 3) | 4.74 ± 0.01 | 0.00 ± 0.00 | 0.00 ± 0.00 |
| Day 7 (Pre-treatment Control) (n = 3) | 5.19 ± 0.04 | 0.00 ± 0.00 | 0.00 ± 0.00 |
| Treatment End | | | |
| Saline Control (IT) (n = 6) | 5.20 ± 0.03 | 3.24 ± 0.08 | 3.63 ± 0.34 |
| Clofazimine (Oral) 20 mg/kg (n = 6) | 3.95 ± 0.03 | 3.14 ± 0.03 | 3.01 ± 0.03 |
| Composition of Example 1 (Sus.) | 3.43 ± 0.18 | 2.93 ± 0.25 | 3.10 ± 0.02 |
| (IT, aerosol) 10.0 mg/kg (n = 6) | | | |

The data in Table 7 indicate that treatment with the composition of Example 1 led to the greater reduction in bacterial recovery in the lungs and spleen of animals infected with M. avium.

### Chronic Beige Mouse Model

In this experiment, 6 to 8 week-old Beige mice were rested one week before infection. Mice received a pulmonary infection of 1×10⁸ CFU of M. avium 2285 rough on Day 0. Three mice were sacrificed on Day 1, and six mice on Day 27 to determine bacterial uptake and pre-treatment bacterial loads. Whole lungs, spleens, and livers were extracted, homogenized in 4.5 ml of 1×PBS and plated at (0-1-2-3-4-5-6-7) dilutions on 7H11 and charcoal agar plates. The plates were placed in a 37° C. dry-air incubator for 25 to 30 days.

The remaining infected Beige mice were treated every other day, starting on Day 28, for a total of 14 treatments. Animals received one of the following treatments: Saline (Microsprayer®, 35 μl); Clofazimine (oral gavage, 20 mg/kg, 200 μl); Composition of Example 1 (IT, Microsprayer®, 10 mg/kg, 35 μl).

Mice were sacrificed on Day 57, two days after the final treatment. Plates were placed in a 37° C. dry-air incubator for 30 days.

Statistical analysis was performed by first converting CFU to logarithms, which were then evaluated by a one-way ANOVA followed by a multiple comparison analysis of variance by a one-way Tukey test. Differences are considered significant at the 95% level of confidence.

Average Log₁₀ CFU data following Beige mouse M. avium chronic infection are shown in Table 8.

TABLE 8

| | Lung | Spleen | Liver |
|---|---|---|---|
| | Log₁₀ CFU ± | Log₁₀ CFU ± | Log₁₀ CFU ± |
| Group | SEM | SEM | SEM |
| Day 1 (Pre-treatment Control) (n = 3) | 5.24 ± 0.05 | 0.00 ± 0.00 | 0.00 ± 0.00 |
| Day 27 (Pre-treatment Control) (n = 6) | 6.02 ± 0.07 | 5.37 ± 0.35 | 6.52 ± 0.12 |
| Treatment End (Day 57 | | | |
| Saline Control (IT) (n = 6) | 6.40 ± 0.06 | 6.15 ± 0.16 | 6.69 ± 0.14 |
| Clofazimine (Oral) 20 mg/kg (n = 6) | 5.94 ± 0.12 | 3.08 ± 0.04 | 4.07 ± 0.04 |
| Composition of Example 1 (Sus.) (IT, | 2.45 ± 0.09 | 4.83 ± 0.22 | 3.89 ± 0.011 |
| aerosol) 10.0 mg/kg (n = 6) | | | |

These data suggest that clofazimine has a difficult time penetrating the granuloma-like structures formed by established, “chronic” animal NTM infection models. It appears that the composition of the present invention does not have the same issues, and is able to maintain antimycobacterial activity even after the infection has become well-established.

### Effect of the Composition of Example 3 on Barrier Integrity and Inflammation Following Exposure to Pulmonary Epithelial Cells In Vitro

Cell Viability: Three different cell types under two in vitro conditions were used to assess pulmonary epithelial cell viability: Calu-3; A549; and hAELVi cells. Cells were either treated under “submerged conditions” (i.e. in cell culture media on Transwell™ plates) or “air-liquid interface” mimicking conditions (ALI), which had cell culture media removed from the apical side of the cells. In “submerged conditions”, Calu-3 cells were exposed to three doses of the Composition of Example 3 (10%, 50%, or 100%) for four hours. To estimate cell viability, cells were stained using acridine orange/propidium iodide (AO/PI) staining to differentiate live/dead cells. Red fluorescence signaled cell death.

Macrophage Uptake: THP-1 cells were differentiated to macrophage-like cells following incubation with 124 ng/ml phorbol 12-myristate 13 acetate (PMA) for 3 days. Once the cells were matured, they were exposed to the Composition of Example 3 (diluted 1:200 in Hank's Buffered Salt Solution (HBSS)) for four hours. Cells were stained via AO/PI, as described above, to determine cell viability following exposure.

### Transepithelial Electrical Resistance (TEER) Measurements

Calu-3 cells were seeded at 1×10⁵ cells/well on a Transwell™ 3460, and left for 12 days to grow to confluence. TEER measurements were performed using an EVOM2 (World Precision Instruments, Friedberg, Germany) according to the manufacture's instructions. Following seeding, Calu-3 cells were exposed to either saline (negative control) or the Composition of Example 3 (concentrations: 20 mg/ml, 10 mg/ml, or 2 mg/ml). The cells were exposed from 2 to 4 hours, before measuring TEER.

### Inflammatory Cytokine Production

Differentiated THP-1 cells (dTHP-1) were exposed to the Composition of Example 3 for 4 hours or 24 hours (1:200 HBSS dilution). HBSS exposure alone was used as a negative control, and lipopolysaccharide (LPS) (100 ng/ml) was administered as a positive control.

Following the incubation, supernatant was removed from the cells t=4 hours or 24 hours, and the pooled. An enzyme linked immunosorbent assay (ELISA) was performed on the pooled supernatant samples. Individual ELISA kits for TNF-α, IL-6, IL-8, and IL-10 were used, according to the manufacturer's instructions.

Statistical analysis was performed by one-way analysis of variance (ANOVA) followed by Tukey post-hoc test. Statistical significance was determined at a probability value <0.05.

Results: Under “submerged” conditions the Composition of Example 3 led to no visual reduction in cell viability over four-hour incubation at any of the concentrations administered.

Under “ALI” conditions, three different cell types (Calu-3, A549, and HAELVi cells) were investigated over three different time points (five hours, two days, and seven days). The was little to no cytotoxicity observed at four hours in any of the cells, or at day 2 in the Calu-3 cells. Some toxicity was seen at day 2 and day 7 in A549 cells, and day 7 in Calu-3 cells. Technical limitations did not permit quantification of cell death.

With regard to macrophage uptake, differentiated THP-1 cells were incubated at 1:200 HBSS for four hours to determine macrophage cell viability after exposure. The Composition of Example 3 did not induce cell death, but did show clofazimine uptake by the macrophages.

With regard to TEER measurements, Calu-3 cells were exposed to HBSS or three concentrations of the Composition of Example 3 for four hours, and TEER measurements were sampled at various time points throughout the exposure. A reduction in TEER of ≥50% compared to controls at any given time point was considered a significant loss in barrier integrity.

Exposure to the Composition of Example 3 to Calu-3 cells had no effect on barrier integrity after one-hour exposure. Exposure at 20 mg/ml led to significant (i.e. ≥50%) reduction after two hours. At a concentration of 10 mg/ml showed a slight reduction (i.e. 25-35%) at all time points after two hours. Exposure at 2 mg/ml did not show any reduction in barrier function over the full study duration.

### Inflammatory Cytokine Production

The positive control LPS behaved as expected in this model. The Composition of Example 3 demonstrated no significant changes in cytokine at any timepoint investigated. Results are shown in Table 9.

Cytokine Production Following dTHP-1 Cell Exposure

TABLE 9

| | Cytokine concentration (pg/ml) | | | |
|---|---|---|---|---|
| | TNF-α | IL-8 | IL-10 | IL-6 |
| Treatment Group | (4 hr) | (4 hr) | (4 hr) | (24 hr) |
| Control | 603.0 ± 23.0 | 398.3 ± 12.0 | 16.5± | 7.9 ± 0.3 |
| LPS | 2130.7 ± 23.0* | 2596.8 ± 84.0* | 65.1 ± 7.0* | 22.6 ± 0.7* |
| Composition of Ex. 3 | 441.9 ± 48.3 | 978.9 ± 395.4 | 18.9 ± 7.1 | 6.5 ± 1.4 |

(*p < 0.05)

### In Vivo Safety and Tolerability

In this study, 6-8-week-pld Balb/C female mice were given a total of three doses every other day. Mice were dosed at 10.0, 5.01, and 2.51 mg/kg using the Composition of Example 1. The composition was given via Microsprayer® aerosol intratracheal (IT) administration, at volumes of 35 μl/mouse. Following instillation, the mice were observed at 10 minutes, 1, 2 and 4 hours after dosing, and then daily afterwards.

Table 10 shows gross observations following administration. “BAR” indicates the animals were bright, active and responsive.

TABLE 10

| Treatment Group | Day 1 | Day 2 | Day 3 |
|---|---|---|---|
| Group 1 | BAR | BAR | BAR |
| 10.0 mg/kg | | | |
| Group 2 | BAR | BAR | BAR |
| 5.01 mg/kg | | | |
| Group 3 | BAR | BAR | BAR |
| 2.51 mg/kg | | | |

Table 11 shows weights of the animals over the three days tested.

TABLE 11

Weight (grams)

| Mouse | Dose | Day 1 | Day 2 | Day 3 |
|---|---|---|---|---|
| 1 | 10 mg/kg | 20.1 | 20.2 | 22.1 |
| 2 | ″ | 21 | 21.2 | 22 |
| 3 | ″ | 21.1 | 20.2 | 22.2 |
| 1 | 5.01 mg/kg | 22.3 | 22.3 | 23.1 |
| 2 | ″ | 22.1 | 22 | 23.2 |
| 3 | ″ | 22 | 21.9 | 23.2 |
| 1 | 2.51 mg/kg | 22 | 22 | 23.3 |
| 2 | ″ | 22.2 | 22.2 | 22.8 |
| 3 | ″ | 22.4 | 22 | 23.1 |

These data indicate that there was no statistically significant change in body weight over the three treatment days. These results indicate that compositions of the present invention are well tolerated at the doses tested.

### Example 4

### Toxicokinetics Study Using Clofazimine Inhalation Suspension (CIS) for the Treatment of Pulmonary NTM Infection in Canine Model.

In this study, clofazimine inhalation suspension was evaluated in canines to determine toxicity over 28 days of once-a-day dosing regimen. The animals were studied for a period of 84 days. Using the recommended good laboratory procedures (GLP) guidance, a repeat dosing study evaluated low, mid and high dosing of clofazimine (at 2.72 mg/kg and 2.95 mg/kg (low); 5.45 mg/kg and 5.91 mg/kg (mid); and 10.87 mg/kg and 10.07 mg/kg (high) to average male versus female.

### Clofazimine Inhalation Suspension (CIS) for Nebulization

The clofazimine inhalation suspension (CIS) formulation consists of clofazimine particles (20 mg/mL) suspended in 0.9% saline, with polysorbate 80 (0.5% v/v) to stabilize the suspension. The drug product is stable at room temperature for 1 year. Vehicle consisted of sterile 0.5% Polysorbate 80 (Hx2) in 0.9% saline. The formulation was optimized and tested in vitro at PharmBioTec to reduce animal experiments (data not shown).

Aerosolized/Nebulized clofazimine formulation as prepared above, was administered to the animals over a period of 30, 60, and 120 minutes using a jet nebulizer and samples collected for evaluation and analysis as described below.

### Study Design

All GLP canine study activities were performed by Lovelace Biomedical Research Institute (LBRI) under an IACUC approved protocol. Five groups of Beagle dogs were divided into study groups (Table 1) and exposed once daily by face-mask inhalation to filtered air (Group 1), vehicle (Group 2-0.5% polysorbate in saline), or CIS (Groups 3-5) for 28 consecutive days. The Low, Mid, and High doses (Groups 3, 4, and 5) were exposed to an average aerosol concentration of 0.209 mg/L for 30 min, 60 min and 120 min, respectively. The 28-day exposure period was followed by 28- and 56-day no exposure recovery periods (Recovery Study, Study Day (SD) 56 and 84 respectively). Blood was collected for toxicokinetic (TK) analysis from animals after both the first day of dose administration (Day 1; (0.5 hours (±5 minutes), 6 hours (±30 minutes), 12 hours (±30 minutes), 24 hours (±1 hour)), the last day of dose administration (SD28), and at SD41, SD56 and SD84, as indicated in Table 1. Main Study animals were necropsied one day post final exposure (SD29), while two male and two female Recovery Study animals were each necropsied on SD56 and SD84.

TABLE 12
Mean toxicokinetic parameters in dog
plasma following a single dose on study day 1, or 28
consecutive daily doses on study day 28 for low-dose
(group 3), mid-dose (group 4), or high-dose (group 5)
animals

| Study | | | Dose | Cₘₐₓ ± SE | | |
|---|---|---|---|---|---|---|
| day | Group | Exposure | (mg/kg) | (ng/ml) | [symbol] | [symbol] |
| 1 | 3 | Low dose | 0.68 male | 16.2 6 5.42 | 7.19 | |
| | | | 0.74 female | 22.9 6 1.79 | 8.99 6 0.632 | |
| 1 | 4 | Mid dose | 1.36 male | 43.1 6 11.0 | 8.95 6 1.51 | |
| | | | 1.48 female | 33.2 6 6.22 | 9.43 | |
| 1 | 5 | High dose | 2.72 male | 112 6 13.7 | 7.89 | |
| | | | 2.52 female | 139 6 30.1 | 7.23 6 1.03 | |
| 28 | 3 | Low dose | 0.68 male | 27.5 6 5.06 | | NR |
| | | | 0.74 female | 67.1 6 17.7 | | 83.4 |
| 28 | 4 | Mid dose | 1.36 male | 93.5 6 19.9 | | 106 |
| | | | 1.48 female | 183 6 42.9 | | 78.2 |
| 28 | 5 | High dose | 2.72 male | 271 6 68.7 | | 98.1 |
| | | | 2.52 female | 241 6 20.3 | | 115 |

[symbol]

indicates data missing or illegible when filed

### Aerosol Dosing

Aerosolized CIS was delivered to each dog by way of a 6-port aerosol exposure system using three Micro Mist compressed air jet nebulizers. (Supplementary Figure S1) All animals were conditioned to the face masks and restraint system before study. The exposure system and aerosol characteristics of the API and vehicle, including target aerosol concentration, concentration homogeneity, concentration repeatability, and aerosol size distribution were confirmed by LBRI prior to study. Dosing for each dog was measured for every dose delivered throughout study and calculated against individual dog weights.

### Tissue and Plasma Analysis

Blood samples were immediately processed or held on wet ice for no more than 2 hours (hr) before being processed to plasma by centrifugation (1300 g, 2-8° C., ≥10 minutes), with plasma separated into appropriately labeled vials and stored frozen (−70 to −90° C.) until CFZ analysis.

Main study animals (3M/3F per timepoint) had blood samples collected at the following timepoints post-exposure on SD1 and SD28: 0.5 hr (±5 minutes), 6.0 hr (±30 minutes), 12 hr (±30 minutes), and 24 hr (±1 hr). Recovery animals (2M/2F per Recovery timepoint) from each group had blood collected on SD42, and prior to euthanasia on SD56 or SD84. The sample size for each group, sex, and timepoint is described in Table 1. Actual collection times were documented, and all animals in this study underwent scheduled sampling at their respective timepoints.

### LC-MS Determination of CIS

Clofazimine was extracted by protein precipitation from dog plasma. Clofazimine from tissues was first extracted by homogenization of tissues with a Bead Rupter during the extraction process. Reversed-phase HPLC separation was achieved with a Waters Acquity UPLC BEH C18 (2.1×50 mm, 1.7 μm) column on a Shimadzu Nexera X2 UHPLC system. Subsequently, MS/MS detection (Sciex Triple Quad 5500) was set at mass transitions of m/z 473.2→431.1 for clofazimine, and 480.2→432.1 for clofazimine-d7 respectively in positive mode. Retention time and peak area were determined by Analyst® Data Acquisition/Processing Software (Version 1.6.3). Analyte concentrations were obtained from a calibration curve constructed by plotting the peak area versus the nominal concentration using Analyst.

### Pathology

Tissues were collected, examined, weighed as applicable, and representative samples were preserved for histopathology. Eyes with optic nerves, testes, and epididymides were fixed in Modified Davidson's Fluid; other tissues were fixed in 10% neutral buffered formalin (NBF). Lung lobes were instilled via major airway(s) with NBF (to approximate physiologic full lung volume at 25 cm hydrostatic pressure); the major airway(s) used for instillation were closed, and the lung/lobe immersed in NBF for fixation.

Tissues were paraffin embedded, sectioned and stained with hematoxylin and eosin for microscopic examination. Histopathologic examination was conducted in a “read down” fashion: i.e. all tissues and gross lesions were examined for animals exposed to filtered Air Control, Vehicle Control or CIS via face-mask inhalation at the High Dose. Only respiratory tissues (lungs, tracheobronchial lymph node, pharynx, larynx, trachea and nose/turbinates) and gross lesions were examined in Low and Mid Dose animals.

Visceral fatty tissue was evaluated for discoloration during necropsy for each animal. Any skin discoloration was noted by external examination by the attending pathologist during necropsy. Several skin samples were taken from the inguinal region for evaluation. All findings for a given tissue were graded subjectively and semi-quantitatively by a single pathologist on a scale of 1-5 (1=Minimal, 2=Mild, 3=Moderate, 4=Marked, 5=Severe).

### Analysis-

Clinical observations were descriptive while numerical evaluations (means and standard deviations) were used for all other parameters where possible. Toxicokinetic parameters were estimated for blood plasma using Phoenix WinNonlin version 8.3 software (Certara L.P.) with a non-compartmental analysis (NCA) consistent with aerosol administration (extravascular model) on each subject at each timepoint. NCA was only performed if there were quantifiable concentrations at two consecutive timepoints. Concentration values below the lower limit of quantitation of 2.00 ng/ml for plasma were labeled as below quantitative limits (BQL). These BQL values were treated as missing and excluded from calculation of descriptive statistics and toxicokinetic analysis.

Concentrations were used with full precision to three significant figures as received from bioanalytical data. Individual concentrations for clofazimine in plasma were collected by subject and timepoint for males and females separately. The area under the concentration versus time curve (AUC0-24 hr) for each subject on SD 1 and SD 28, from time zero to the timepoint at which the last quantifiable concentration was observed, was calculated with the linear up, log down interpolation method. Parameters were also estimated for time of maximum observed concentration (Tmax), dose normalized AUClast, the maximum observed concentration (Cmax), and dose normalized Cmax. The terminal elimination phase of each concentration versus time curve was identified using at least the final three observed concentration values. The slope of the terminal elimination phase was determined using log regression with uniform weighting. Parameters derived from the terminal elimination phase were reported if they passed the reporting criteria: the coefficient of determination (R2) was greater or equal to 0.8, and the extrapolation of the AUC to infinity was less than or equal to 20% of the total area. The average doses by group and sex, assuming 25% deposition fraction, are transcribed from the aerosol report. In addition, data were averaged for each gender and group to include the timepoints sampled from the recovery period on SDs 42, 56, and 84. Since blood was collected from different animals for the Main Study (SD 1 and SD 28) and Recovery timepoints, individual NCA parameters could not be calculated. Instead, gender/group average concentrations were used to include both sets of data.

### Statistics

Arithmetic mean, standard deviation (SD), and sample size of clofazimine concentrations in plasma were calculated for each group and timepoint in Phoenix for males and females separately. Coefficient of variance (CV %) was calculated for NCA parameters, and geometric mean and geometric SD were calculated for accumulation ratios in Phoenix. Sex ratios were calculated in Phoenix by dividing dose-normalized AUClast and dose-normalized Cmax for male animals by the same parameters for female animals. Accumulation ratios were calculated in Phoenix by dividing dose-normalized AUClast and dose-normalized Cmax after 28 consecutive daily doses (Day 28) by the same parameters after a single dose (Day 1) for male and female animals. Dose-proportionality ratios were calculated in Phoenix by comparing dose groups pairwise for dose-normalized AUClast and dose-normalized Cmax values after 28 consecutive daily doses (SD28) or after a single dose (SD1) for male and female animals.

The dose-dependence of Cmax and AUC0-24 was evaluated as a power-law (i.e., Cmax, AUC0-24˜Dosen) was evaluated for pooled data. The effects of sex, treatment day, and interactions between sex and treatment day were estimated by multiple linear regression on log-transformed coordinates. A full model was run to identify significant terms (p<0.05) and the reduced model was then run to obtain estimates of the parameters. The regression was done in Excel.

### Pulmonary Function and Clinical Observations

There were no CIS related changes in tidal volume, respiratory frequency, or minute volume at any dose level that were statistically significant. Exposure to API produced similar results to the vehicle or air controls. Interdose differences were not considered test article related because they were sporadic, transient, or within expected ranges of variation for beagle dogs undergoing similar study procedures.

There were no abnormal clinical observations reported for any animal at any evaluation point in the study. Body weight trends were analyzed for males and female dogs separately. No specific trend in weight gain or loss was seen due to sex. Weights during the 28-day dosing period showed no specific trend for any dose regimen, for males or females, and in general treated dogs retained a stable weight. Weight changes during the recovery period did not correlate to drug dosing, as both the air and vehicle control weights varied at least as much as the weights of dogs in any of the treatment groups, over the recovery period.

Ophthalmic examination of dogs before and after the 28-day treatment period were unremarkable and did not identify any issues related to treatment. Electrocardiogram results did not indicate any changes to heart rate, PR, QRS, or Qt interval. There were no obvious API related abnormalities in rhythm or waveform morphology at any dose level when compared to the vehicle group and to the pre-dose period.

### Hematology and Clinical Chemistry

Hematology, serum chemistry and urinalysis parameters were obtained at necropsy, on SD29 for animals designated for main study, SD 56 for animals designated for 28-day recovery, and SD84 for animals designated for 56-day recovery. Up through SD29, there were 3 animals per gender/group for all measures, with 2 animals per gender/group for recovery measures at SD56 and SD84.

All notable hematology, clinical chemistry and urinary measures were either unremarkable, incidental, or not considered related to the administration of CIS. No parameter indicated toxicity of any kind, nor followed any dose relationship with CIS.

### Clinical Pathology

At each designated necropsy (SD29, SD56, SD84), tissues were collected, weighed as applicable, and preserved for histopathologic examination. In general, visceral adipose tissue was examined throughout, and discoloration was found only in test article treated animals on SD29. Gross observations related to the test article at the time of necropsy in SD29 (Main Study) animals consisted of mild to moderate, diffuse, yellow discoloration of the adipose tissue in all High Dose animals, two Mid Dose males and all females, and one Low Dose female, but there were no correlating microscopic findings to explain the discoloration. No discoloration of the skin was noted for any animal. Organ weights were collected and analyzed as absolute organ weight, organ to body weight ratio, and organ to brain weight ratio versus the Air and Vehicle Controls. Some organ weight differences were statistically significant in males (lung, adrenal glands, heart, epididymides, testes) and females (adrenal, liver and spleen), but there were no correlating microscopic findings to explain the differences. Remaining organ weights were generally unremarkable relative to Air and/or Vehicle Controls. There was often no consistency across sexes, or ratio measures, and the changes were typically of small magnitude. In addition, there were no test article related observations in any tissue examined.

Histopathological examination of tissues did not determine any finding of significance, with any reported lung or lymph node infiltrates being reported as largely mild or minimal in nature.

### Plasma Toxicokinetics

There were no quantifiable clofazimine plasma concentrations for males or females in the Air or Vehicle control groups (Groups 1 and 2) at any timepoint in the study. Male and female animals in the CIS Low, Mid, and High dose groups had quantifiable clofazimine concentrations at all timepoints sampled on SD1 and SD28. (see Table 13). On SD42 (14 days of recovery from exposure) measurable concentrations were reported for all but one animal that was BQL. On SD56, only one animal had a reportable CFZ concentration while all the others were BQL. All samples were BQL on SD 84.

TABLE 13
Group mean concentrations in male and female dog lung following 28
consecutive daily doses (SD 29) or recovery (SD 56 and 84) from
low dose (group 3), mid dose (group 4), and high dose (group 5)

| Sex | Group | Dose | Study day | | Mean ± SD (ng/g) |
|---|---|---|---|---|---|
| Male | 3 | 0.68 | 29 | 3 | 9450 ± 5940 |
| | | | 56 | 2 | 8600 ± 9190 |
| | | | 84 | 2 | 6320 ± 8030 |
| | 4 | 1.36 | 29 | 3 | 76700 ± 10800 |
| | | | 56 | 2 | 15300 ± 7790 |
| | | | 84 | 2 | 1680 ± 1910 |
| | 5 | 2.72 | 29 | 3 | 186000 ± 84500 |
| | | | 56 | 2 | 92400 ± 67400 |
| | | | 84 | 2 | 27300 ± 7000 |
| Female | 3 | 0.74 | 29 | 3 | 19800 ± 19500 |
| | | | 56 | 2 | 5670 ± 3400 |
| | | | 84 | 1 | 604 ± NA |
| | 4 | 1.48 | 29 | 3 | 62300 ± 33900 |
| | | | 56 | 2 | 83500 ± 20800 |
| | | | 84 | 2 | 8710 ± 10200 |
| | 5 | 2.52 | 29 | 3 | 320000 ± 86700 |
| | | | 56 | 2 | 389000 ± 33200 |
| | | | 84 | 2 | 43100 ± 16900 |

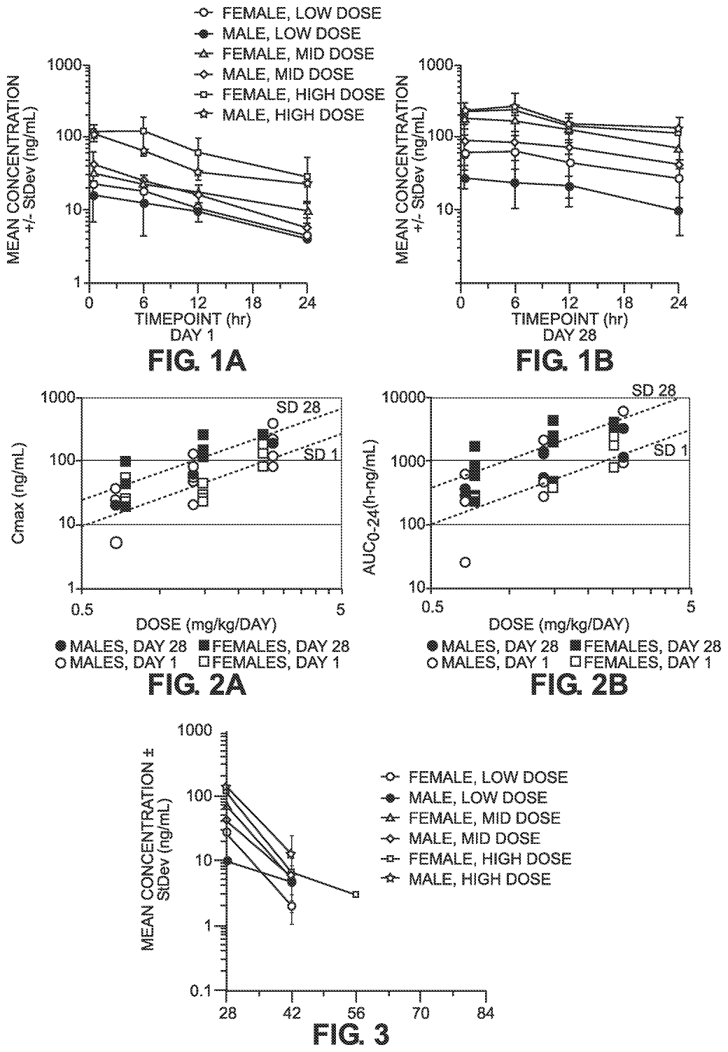

After exposure on Day 1, the time of maximum concentration, or Tₘₐₓ, was at the 0.5 or 6.0 hr timepoint for the Low, Mid, and High dose (Groups 3, 4, and 5, respectively) males and females. After 28 consecutive daily doses on SD28, Tₘₐₓ was at the 0.5, 6.0, or 12.0 hr timepoint for female and male animals in all dose groups. Mean peak clofazimine concentrations (Cₘₐₓ) on SD1 were 16.2 ng/ml, 43.1 ng/ml, and 112 ng/ml for males in the Low, Mid, and High dose groups and 22.9 ng/ml, 33.2 ng/ml, and 139 ng/ml for females in the Low, Mid, and High Dose groups. After 28 consecutive daily doses of CIS, on SD28, mean Cₘₐₓ was 27.5 ng/ml, 93.5 ng/ml, and 271 ng/ml for males in the Low, Mid, and High dose groups and 67.1 ng/mL, 183 ng/ml, and 241 ng/ml for females in the Low, Mid, and High dose groups. (FIG. 1)

Terminal elimination parameters could only be estimated for some animals on SD1 and could not be estimated for any animals on SD28. For animals with reportable terminal elimination parameters on SD1, mean terminal elimination half-life of clofazimine was 7.19 hr, 8.95 hr, and 7.89 hr for males in the Low, Mid, and High dose groups and 8.99 hr, 9.43 hr, and 7.23 hr for females in the Low, Mid, and High dose groups.

Terminal elimination parameters also were calculated with the recovery timepoints of SD42, SD56, and SD84. These terminal half-life values were NR, 106 hr, and 98.1 hr for males in the Low, Mid, and High dose groups, respectively, and 83.4 hr, 78.2 hr, and 115 hr for females in the Low, Mid, and High dose groups, respectively. These are equivalent to a range of 3-5 days for males and females.

TABLE 14
Study groups, exposure, and collection points

| | | | | | [symbol] | | | |
|---|---|---|---|---|---|---|---|---|
| | | | Exposure duration | Deposited lung dose | Main study | | Recovery study | |
| Group | Exposure | [symbol] | (minutes) | 25% DFA (mg/kg) | [symbol] | | [symbol] | |
| 1 | Air control | Not applicable | 120 | | 3 male | 3 female | 4 male | 4 female |
| 2 | Vehicle control | 0.18 mg/L total aerosol | 120 | | 3 male | 3 female | 4 male | 4 female |
| | | [symbol] | | | | | | |
| 3 | Low-dose MNKD-101 | 0.40 mg/L total serozol | 30 | 0.68 M/0.74 F (0.72) | 3 male | 3 female | 4 male | 4 female |
| 4 | Mid-dose MNKD-103 | [symbol] (estimated | 60 | 1.36 M/1,48 F (1.42) | 3 male | 3 female | 4 male | 4 female |
| 5 | High-dose MNKD-101 | 0.209 mg/L clofazimine | 120 | 2.72 M/2.52 F (2.62) | 3 male | 3 female | 4 male | 4 female |
| | | [symbol] | | | | | | |

[symbol]

indicates data missing or illegible when filed

In multiple linear regression on log-transformed variables, treatment on SD28 had the effect of increasing the intercept in the model (p<0.003) but did not affect the slope. The sex of the animal (and interactions) did not significantly affect the intercept or slope of the best-fit model. The mean±SE exponents for Cₘₐₓ (1.44±0.14) and AUC₀₋₂₄ (1.47±0.16) indicate a supra-proportional dose response because the values are not consistent with a hypothesized slope of 1 (p<0.007). The geometric mean ratios of Cₘₐₓ and AUC₀₋₂₄ on SD28 to SD1 were 2.56 (15% CV) and 3.76 (17% CV), respectively (FIG. 2).

Mean accumulation ratios between SD28 (after 28 consecutive daily doses) and SD1 (after a single dose) for CIS DN AUC₀₋₂₄ hr were 5.39, 3.96, and 3.86 for males in the Low, Mid, and High dose groups and 3.80, 6.92, and 2.90 for females in the Low, Mid, and High dose groups. Accumulation ratios for DN Cₘₐₓ were 2.27, 2.54, and 2.44 for males in the Low, Mid, and High dose groups and 3.08, 5.43, and 1.98 for females in the Low, Mid, and High dose groups. All of the ratios indicate ≥2-fold accumulation in males and females after 28 consecutive daily doses. Plasma levels of MNKD-101 quickly decreased from maximum levels (SD28) to BQL for nearly all animals by SD56. (FIG. 3)

### Lung Tissue Toxicokinetics

The toxicokinetics of CIS in lung tissue were evaluated from male and female beagle dogs following 28 consecutive daily doses (SD29) and during the recovery period (SD56 and SD84). There were no quantifiable clofazimine concentrations in the Air or Vehicle Control groups. In general, lung concentration of clofazimine was dose dependent across males and females with measured drug levels following dosing regimens at all time points except one. (Table 14) Female lung drug levels were higher than male levels at SD54 for the mid and high dose groups, but showed no specific trend at SD29 or SD84. Combined sex data clearly shows residency of clofazimine at all timepoints in a dose dependent manner.

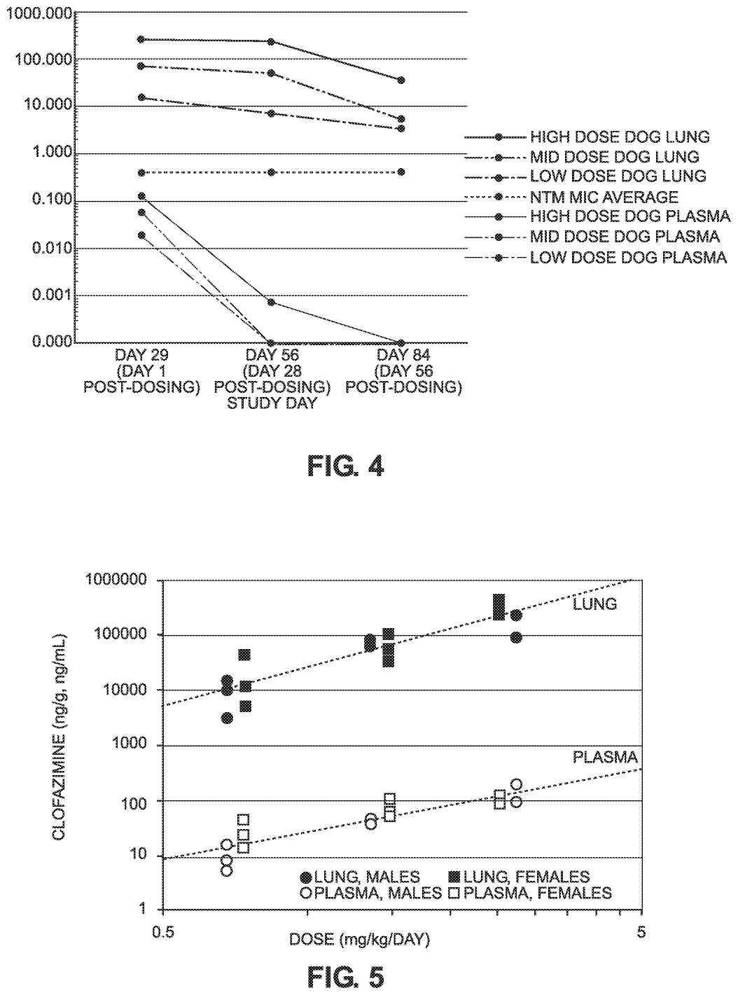

Compared to plasma levels, lung concentrations of clofazimine decreased more slowly from SD29 to SD84 and remained well above the average MIC (minimum inhibitory concentration) for NTM infections. (FIG. 4) Terminal elimination half-lives were estimated only for Low dose females (10.9 days), Mid dose males (9.96 days), and High dose males (19.8 days); other groups did not meet the reporting criteria. Ratios for males compared to females for DN AUClast were near unity for the Low dose groups, and DN AUClast and DN Cmax were near unity for Mid dose groups, indicating no notable (>2-fold) difference between sexes after 28 consecutive daily doses from SD29 to SD84. But Cmax for the Low dose group was nearly 2-fold increased and both DN AUClast and Cmax were more than 2-fold increase for females compared to males in the High dose group, so averages across males and females for toxicokinetic parameters are not presented since males and females were not comparable at all dose levels. Dose-proportionality as assessed by dose-normalized (DN) AUC last and Cmax ratios between dose groups showed much greater than dose proportional increases across dose groups for males and females. Ratios between dose groups for AUC last ranged from 1.57 to 3.44 for males and 2.86 to 8.27 for females. Ratios between dose groups for Cₘₐₓ ranged from 1.21 to 4.91 for males and from 2.11 to 5.76 for females.

As shown in the figures and tables, the experiments showed that a CIS formulation can provide an improvement of residence time, and concentration, within the target organ, the lung, while reducing systemic accumulation, and improved of reduction of systemic toxicity. This GLP toxicokinetic study in beagle dogs provides confirmatory evidence that not only does CIS administration via inhalation reduce systemic clofazimine accumulation, and non-target organ toxicity, but also leads to superior deposition in the lung, at levels above the average MIC for NTM infections. Lung clofazimine levels remained at concentrations well above the NTM MIC even 56 days post dosing, while systemic exposure to clofazimine remained low, indicating that no reserve pools of drug were coming from tissue accumulation. This is an important finding that supports the observations that there were no obvious or measurable adverse effects from drug accumulation. For example, the fact that no animal was reported to have any skin discoloration is promising in terms of reducing a primary adverse effect of clofazimine administration in humans, skin discoloration. Importantly, clearance of CIS was also not dependent upon dose received, indicating that clearance mechanisms were not saturated by the dose levels used. This is clearly indicated by the fact that t_{1/2} showed no trends in any direction across dose groups, indicating that administering even higher doses of clofazimine may be possible without untoward effects.

Dose levels used in this study were determined from prior studies in mice and rats at doses up to 3.45 mg/kg/day, with a rat study also demonstrating very poor clofazimine deposition in the lung from oral administration. Interestingly, the total lung fraction of clofazimine in the low dose group after 28 days of dosing appears to be primarily due to simple accumulation, while the highest dosing accumulation (exponential increase) indicates reduced transport of clofazimine out of the lung, likely by incorporation into the lung resident macrophage population. This is particularly relevant for patients with reduced lung function from co-morbidities that may result in irregular deposition of CIS. Combined with its very small, inhaled particle size (1.5-2.5 MMAD) that reach deep within the lung, CIS delivered through the lungs had an increased residence time of clofazimine through macrophage uptake may be a balancing factor against the possibility of irregular deposition from lung abnormalities. Concentrations of clofazimine in plasma and lung tissue at necropsy illustrate the supra-proportional behavior of the PK (FIG. 5). Even at the highest level of clofazimine delivered, no untoward physiology was seen in lung tissues directly, and few adverse events were seen systemically. At all doses, both liver and spleen showed no more than 0.1 microgram clofazimine per gram of tissue only 28 days post-dosing, while adipose tissue showed a sharp linear decline to BQL by SD56, demonstrating the rapid clearance of clofazimine from non-lung tissues.

In summary, all three dose levels showed significant residual drug in lung tissue, demonstrating impressive lung loading and long lung residence time for clofazimine. Drug concentrations in the lung remained well above the average NTM MIC at all timepoints, with measurable clofazimine levels at 28 and 56 days post-dosing. In contrast, plasma levels of clofazimine were consistently measurable only through 14 days post-dosing, with measurements below the limit of quantitation at 56 days post-dosing. The data showed that clofazimine inhalation suspension can provide an effective therapy for the treatment of NTM infections through direct delivery of antibiotics to the lungs, overcoming the systemic toxicity seen in oral clofazimine treatment for NTM.

### Example 5

### Safety and Tolerability of Clofazimine Delivered by Nebulization and Pharmacokinetic (PK) Study in Healthy Subjects

This study design encompassed the first-in-human inhalation study, randomized, double-blind, placebo-controlled study of clofazimine in healthy adult participants, which entailed single and daily repeated doses for 7 days of clofazimine up to 90 mg. Clofazimine inhalation suspensions as prepared above to a concentration of 20 mg/mL was used in this study. The clofazimine composition was provided as a red/orange micronized suspension comprising polysorbate 80 (0.5% v/v), sodium chloride (0.9% wt/v) and water. For placebo, subjects were treated with a nebulized sterile, isotonic saline solution consisting of 0.9% wt/v sodium chloride. Subjects were treated with 30 mg, or 60 mg of a suspension comprising clofazimine for the study period as required with monitoring for adverse events, such as dysphonia, oropharyngeal pain, and cough while taking the drug, and blood samples for laboratory assessment were taken at various intervals during the study period. The doses were administered by inhalation with a Pari e-Flow nebulizer system to investigate tolerability and pharmacokinetics (PK) in healthy human subjects. The subjects were monitored for any life-threatening or severe adverse events and samples of their blood were taken for analysis. Prior to each dose increase, interim safety and PK assessments were conducted, considering both local (pulmonary) and systemic effects, as well as to ensure systemic drug levels being below preclinical no-observed-adverse-effect-level (NOAEL). A 90 mg dose of clofazimine in suspension was also studied in healthy individuals.

In a portion of the study, 24 adults were enrolled in one of three cohorts (n=8 per cohort) that received a single inhaled dose of 30 mg, 60 mg, or 90 mg clofazimine, respectively. Participants resided at the clinical research unit until day 5 post-dose, during which time they were evaluated for safety and samples were collected for PK assessment. Participants returned on days 8 and 15 for additional safety assessments and sample collection. During the MAD portion of the study, 16 adults were enrolled in one of two cohorts (n=8 per cohort) that received a daily inhaled dose of 30 mg or 90 mg clofazimine for a seven-day period. Participants resided at the clinical research unit until day 8 post-dose, during which time they were evaluated for safety and samples were collected for PK assessment. Participants returned on days 15 and 36 for additional safety assessments and sample collection.

Inhaled, nebulized clofazimine was well tolerated with no serious adverse-effects (SAEs) occurring and most adverse events were mild. Subject showed no abnormal, clinically significant electrocardiogram, ECG results were reported. Parameters affected by abnormal, not clinically significant ECG results were the QRS interval and the PR interval. Subjects treated with nebulized clofazimine had a Columbia Suicide Severity Rating Scale (C-SSRS) that showed no suicidal ideation or behavior during the study, and there was no evidence of skin discoloration.

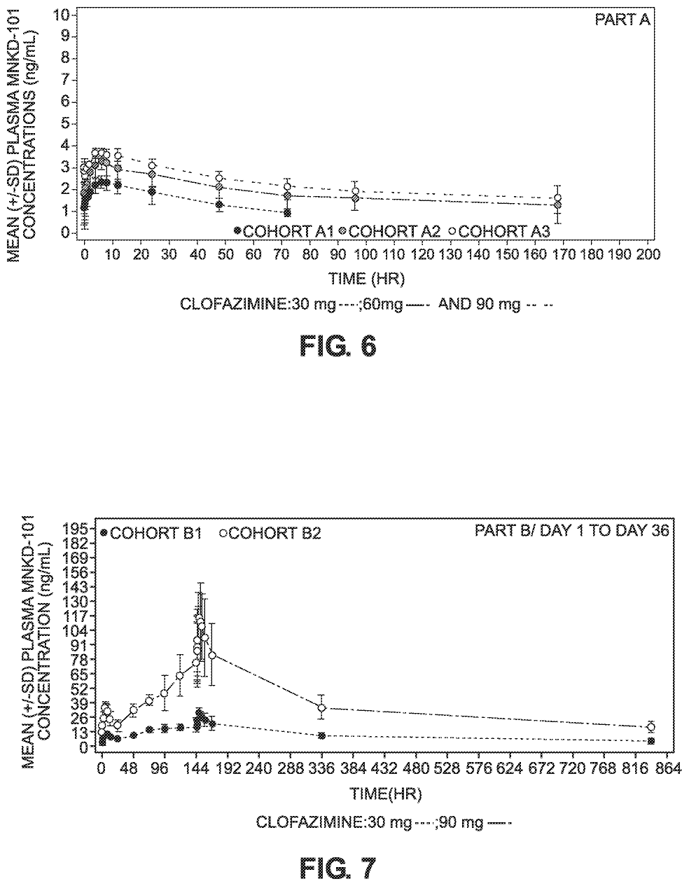

The clofazimine doses studied and used, both in single and repeated administration showed proportional increases in clofazimine plasma levels with increasing dose in human subjects. FIG. 6. Peak plasma concentrations of clofazimine with single administration were reached within 4-8 hrs, whereas with repeated administration this was reached within 2-12 hrs. Dose increases within single administration of clofazimine were associated with a 2.3-fold increase in AUC₀₋₂₄ with a dose increase of 2-fold, and a 1.6-fold increase in AUC₀₋₂₄ with a dose increase of 1.5-fold. The data indicated that clofazimine showed a long plasma half-life of 290 hrs after repeated administration (FIG. 7). FIG. 7 also shows that accumulation following repeated clofazimine dosing was considered meaningful. Cough levels did not appear to plateau between Day 1 and Day 6 which reflects the longer t½. The safety and PK data profiles presented herewith indicate that the clofazimine compositions for inhalation by nebulization are safe to use in humans for the treatment for NTM infections, and a Phase 2/3 efficacy study in NTM (nontuberculous mycobacteria) lung infection is in preparation.

### Example 6

In this study, subjects diagnosed with NTM were treated with a suspension comprising nebulized clofazimine or a placebo using a Pari eFlow® nebulizer to evaluate the safety of treatment. Clofazimine inhalation suspensions as prepared above to a concentration of 20 mg/mL was used in this study. The clofazimine composition was provided as a red/orange micronized suspension comprising polysorbate 80 (0.5% v/v), sodium chloride (0.9% wt/v) and water. For placebo, subjects were treated with a nebulized sterile, isotonic saline solution consisting of 0.9% wt/v sodium chloride. Subjects were treated with 30 mg, or 60 mg of a suspension comprising clofazimine for the study period as required with monitoring for adverse events, such as dysphonia, oropharyngeal pain, and cough while taking the drug, and blood samples for laboratory assessment were taken at various intervals during the study period. Subjects were prohibited from using any inhaled antibiotics with activity against NTM from 28 days before the start of the study, then treated for 28 days once daily. Patients were monitored for any life-threatening or severe adverse events and samples of their blood and sputum were microbiologically analyzed for the presence of bacteria.

Positive therapeutic effects is determined by sample sputum cultures of a patient being negative for NTM bacterium after 3 consecutive sputum cultures each at least two weeks post treatment apart at the end of six months period.

Analysis of the blood samples and sputum from animal studies indicate that the clofazimine inhalation treatments at all doses are modeled to be effective against NTM bacterial infection as described above and the minimum inhibitory concentration (MIC) are exceeded at highest therapeutic doses administered at 90 mg/day. The data indicates that the nebulized clofazimine compositions were determined to be well-tolerated in humans with reduced or minimum adverse effects.

### Example 7

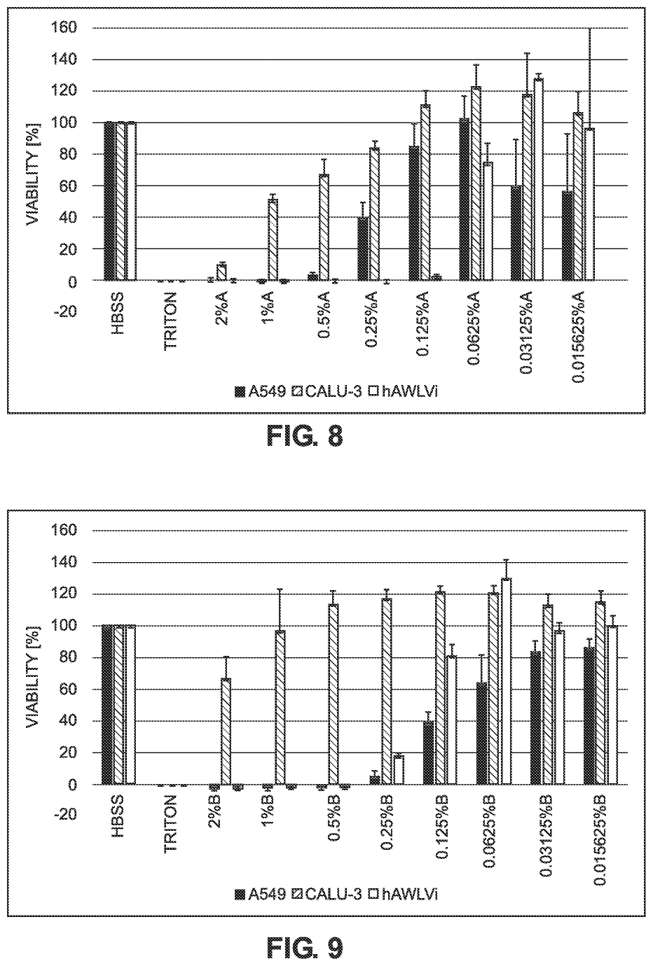

Effects of surfactants on cell viability study: The effects of a surfactant such as polysorbate 80 (PS 80) containing various fatty acids components were used in this study to determine their effect on cell viability in the lungs upon administration of composition for lung delivery. In vitro experiments were carried out using three different human cell lines, i.e., human cell lines A549 (type II pulmonary epithelial cell derived), hAELVi, (human alveolar epithelial cell) and Calu-3 (lung adenocarcinoma cell). The cell lines were obtained from ATCC (Bethesda, MD) and maintained as recommended by the provider and seeded and incubated for the 24 hour test in 96-well plates containing 4×10⁴/well in RPMI 1640 medium (Gibco). For testing, the medium as aspirated, the cells were washed twice with Hank's balanced Salt Solution (HBSS) at pH 7.4 and a 0.2 ml sample solution containing a surfactant was added to the cells. HBSS was used as negative control as well as HBSS containing 1% TritonX-100 as positive control. Surfactants under study were two, a super-refined grade PS 80 comprising a mixture of various fatty acid esters of about 70% to 85% total content being oleic acid and having other components including, mystiric, palmitic, palmitoleic, stearic, linoleic and linolenic acid esters. This super-refined PS 80 (Tween® 80, A) manufactured by Mallinckrodt Baker or Merck, and a second PS 80 (B, NOF Corporation) comprising an oleic acid component of about 99% were tested. In the experiments to determine the in vitro IC₅₀ values, the cell lines were exposed to equal concentrations of the two surfactants in HBSS solution at the same concentrations and several dilutions of the test compounds after incubation for 4 hours at 37° C. with shaking, washed with HBSS. Cell viability was assessed based on the absorbance measurements at 550 nm obtained using the MTT assay as described in Metz et al., J. ALTEX, Jan. 29, 2020: V.2 doi:10.14573/altex.1910231. FIGS. 8 and 9, and Table 16 illustrate data obtained from the experiments.

FIG. 8 and FIG. 9 depict graphs illustrating comparisons between surfactants used in experiments of in vitro cell survival for each of the cell lines used. As illustrated in the graphs, cells treated with HBSS were 100% viable. Cells treated with HBSS containing 1% Triton X-100 loss all viability in the experiments. The effects of the test surfactants on the cell viability were less pronounced at lower surfactant concentration in the medium and cell line type. However, the experiment showed that the surfactant with higher oleic acid concentration PS 80 (FIG. 9) had better cell viability effects, or was less toxic to the cells than the super-refined PS 80 (FIG. 8).

The data also demonstrate that the Calu-3 cells are less sensitive to the concentration of surfactant used than the two other cell lines used since these cells are tumor-derived and expected to behave differently.

TABLE 16

| Substance | A549 cal. IC₅₀ in vitro (%) | Calu-3 calc. IC₅₀ in vitro (%) |
|---|---|---|
| PS 80 (A) | 0.147 ± 0.017 | 1.117 ± 0.071 |
| PS 80 (B) | 0.118 ± 0.007 | 0.224 ± 0.030 |

### Example 8

A study of a clofazimine composition of the present invention administered to healthy adults via a vibrating mesh nebulizer was undertaken. Single and multiple ascending doses up to 90 mg were investigated. The results indicate a rapid and deep lung delivery (8-15 minutes/treatment) with high lung deposition rates of at least 30%. Dose proportional increases in Cₘₐₓ and AUC₀₋₂₄ were seen for single and multiple doses with meaningful dose-dependent accumulation ratios. Half-life was seen to be extended in single and multiple dose studies. Calculations indicate pulmonary clofazimine levels to remain above NTM-MICs (nontuberculous Mycobacterium-minimum inhibitory concentrations) for at least 56 days post treatment course.

Although the foregoing refers to particular preferred embodiments, it will be understood that the present disclosure is not so limited. It will occur to those of ordinary skill in the art that various modifications may be made to the disclosed embodiments and that such modifications are intended to be within the scope of the present invention.

What is claimed is:

1. An inhalable pharmaceutical composition comprising clofazimine, or a pharmaceutically acceptable derivative of clofazimine, a clofazimine salt, or a polymorph of clofazimine, or combination thereof, and a pharmaceutically acceptable carrier and/or excipient for use in the treatment or prophylaxis of a nontuberculous bacterial infection of the lungs, wherein clofazimine is in an amount of 1 mg to 20 mg wt % in the composition, and wherein the inhalable pharmaceutical composition is provided by inhalation in an effective daily dose of up to 90 mg of clofazimine.

2. The inhalable pharmaceutical composition of claim 1, wherein the nontuberculous bacterial infection of the lungs treated or prophylactically dissuaded is caused by a Mycobacterium selected from the group consisting of: Mycobacterium avium, Mycobacterium intracellulare, Mycobacterium abscessus, and Mycobacterium leprae, and a combination thereof.

3. The inhalable pharmaceutical composition of claim 1, wherein the nontuberculous bacterial infection is an opportunistic infection, selected from the group consisting of: Mycobacterium avium complex pulmonary disease and opportunistic nontuberculous infection, or combination thereof, concomitant with one or more of the group consisting of: cystic fibrosis, chronic obstructive pulmonary disease or acquired immune deficiency syndrome.

4. The inhalable pharmaceutical composition of claim 3, wherein the infection is an opportunistic nontuberculous mycobacteria infection in a patient with cystic fibrosis.

5. The inhalable pharmaceutical composition of claim 1 wherein the infection treated or prophylactically dissuaded is caused by mycobacteria or other gram positive bacteria, and is administered by inhalation, before, simultaneously, or subsequent to the administration of an agent selected from the group consisting of: bedaquiline, or a pharmaceutically acceptable salt of derivative thereof, cefoxitine, amikacin, clarithromycin, pyrazinamide, rifampin, moxifloxacin, levofloxacin, and para-amino salicylate, and mixtures thereof.

6. The inhalable pharmaceutical composition of claim 1, wherein the clofazimine is at least about 90% orthorhombic polymorph III.

7. The inhalable pharmaceutical composition of claim 6 wherein such composition is used to treat or as a prophylaxis against a nontuberculous bacterial infection of the lungs.

8. The inhalable pharmaceutical composition of claim 1, wherein the composition is delivered by inhalation for treatment or prophylaxis with a high lung deposition rate of at least about 30%.

9. An inhalable pharmaceutical composition comprising clofazimine, or a pharmaceutically acceptable derivative of clofazimine, a clofazimine salt, or a polymorph of clofazimine, or combination thereof, and a pharmaceutically acceptable carrier and/or excipient for use in the treatment or prophylaxis of a nontuberculous bacterial infection of the lungs, wherein clofazimine is in an amount of 1 mg to 20 mg wt % in the composition, and is delivered by an inhaler configured to cause high lung deposition rates of at least 30%.

10. The inhalable pharmaceutical composition of claim 1, wherein said clofazimine, or a pharmaceutically acceptable derivative of clofazimine, a clofazimine salt, or a polymorph of clofazimine, comprises clofazimine.

11. The inhalable pharmaceutical composition of claim 1, wherein said clofazimine, or a pharmaceutically acceptable derivative of clofazimine, a clofazimine salt, or a polymorph of clofazimine, comprises a pharmaceutically acceptable derivative of clofazimine.

12. The inhalable pharmaceutical composition of claim 1, wherein said clofazimine, or a pharmaceutically acceptable derivative of clofazimine, a clofazimine salt, or a polymorph of clofazimine, comprises a clofazimine salt.

13. The inhalable pharmaceutical composition of claim 1, wherein said clofazimine, or a pharmaceutically acceptable derivative of clofazimine, a clofazimine salt, or a polymorph of clofazimine, comprises a polymorph of clofazimine.
